(19) United States
(12) Patent Application Publication
(10) Pub. No.: US 2018/0160943 A1
(43) Pub. Date: Jun. 14, 2018

(54) SIGNATURE BASED MONITORING SYSTEMS AND METHODS

(71) Applicant: 4iiii Innovations Inc., Cochrane, CA (US)

(72) Inventors: Kiplinig Fyfe, Cochrane (CA); Caitlin Milne, Vancouver (CA); Darren Zacher, Calgary (CA); Tom Williams, Edmonton (US); Victoria Brilz, Cochrane (CA); Vipin Bakshi, Calgary (CA)

(21) Appl. No.: 15/102,262

(22) PCT Filed: Dec. 10, 2014
(86) PCT No.: PCT/IB2014/003114

§ 371 Date: Jun. 6, 2016

(30) Foreign Application Priority Data
Dec. 10, 2013 (US) 61914233

(51) Int. Cl. A61B 5/11; A61B 5/00; A61B 5/145; A61B 5/0488; A61B 5/0476; A61B 5/08; A61B 5/024; A61B 5/01; A63B 24/00

(52) U.S. Cl. CPC A61B 5/1118; A63B 2225/50; A61B 5/7246; A61B 5/443; A61B 5/14542; A61B 5/1112; A61B 5/0488; A61B 5/0476; A61B 5/0816; A61B 5/0024; A61B 5/4875; A61B 5/02405; A61B 5/01; A61B 5/112; A61B 5/14532; A61B 5/742; A61B 5/746; A63B 24/0062; A61B 5/7267; A61B 5/1123; A61B 2560/0228; A61B 2503/40; A61B 2503/10; A63B 2024/0012; A63B 2220/40; A63B 2220/05; A63B 2230/06; A63B 2230/10; A63B 2230/202; A63B 2230/50; A63B 2230/75; A61B 5/7282

(57) ABSTRACT

A system and a method determine an activity of a user. Sensor data is collected from a sensor within a pod worn by the user. The sensor data is matched to a signature definition corresponding to a known activity and the sensor. When the sensor data matches the signature definition, the activity of the user is determined. Sensed data, signature data, and/or matched signature data may be communicated to and from external devices. Signatures may be learned for known activities.

### RELATED APPLICATIONS

This application claims priority to U.S. Patent Application Ser. No. 61/914,233, filed Dec. 10, 2013, and incorporated herein by reference.

### BACKGROUND

Sensors and sensor units have been used to collect performance information of a user. Typically, a sensor is coupled with a processor and a battery to allow independent collection of the performance data. The data may be stored within the sensor unit for later retrieval or transmitted to a data processing unit (e.g., a main computer or server). Raw data from the sensor is typically processed to reduce the size and/or identify a specific feature or event within the captured data.

Signature analysis may also be used to identify a state of a person or a device based upon the sensed data. For example, analysis of data collected from a sensor associated with a person against predefined signatures may determine a state of that person (e.g., accelerometers attached to the person may be used to determine whether the person has fallen).

### SUMMARY OF THE INVENTION

A system stores a series of measurements from sensors and/or fusion of sensors, matches subsets of the stored sensor data to one of any number of pre-determined or learned signatures, calibrates the state change associated with each matched signature against a measured truth, and then using, refining, and sharing the calibrated state change estimates. Sensor fusion combines two or more sensor measurements to get (a) more information about the state (e.g., heart rate and bike cadence give us more information about how the athlete is performing), (b) more robust information about the state (e.g. if we have inertial system and Global Navigation Satellite System (GNSS), the inertial system may help when in GNSS derived environments), and (c) complementary information about the state (again with the GNSS/inertial example; inertial gives us high frequency information, GNSS gives good accuracy low frequency information). For example, the “more information” may result from fusion of sensors that collect disparate information. The pod includes one or more sensors, a microprocessor, and wireless communication capabilities, and is worn on the body of a user or attached to a piece of equipment used by the user. A pod management software system (PMSS) then matches data from the sensors to one or more signature definitions, where a match indicates a particular activity of the user. One or more multicoloured LEDs, a vibrator motor and/or audio codec may be used to generate an alarm and/or to notify the user.

A portable pod attaches to a user's body, a piece of utilized equipment (e.g. golf club, soccer ball, racket, etc.), or a vehicle to monitor the activity of the user. The pod includes one or more sensors that detect activity and/or status of the user or vehicle. The pod includes a signature engine that analyzes data from the sensors against one or more signatures of known activities and states. Each signature may be based upon one or more types of sensor. By identifying the signature that matches the data, the pod determines the activity of the user (or equipment/vehicle).

Signatures may be validated and/or calibrated based upon determined direct truth measurements that define one or more parameters of an activity accurately.

Signatures are stored in a database on a server and may be loaded into the pod based upon expected activity of the user.

In one embodiment, a method determines an activity of a user. Sensor data is collected from a plurality of sensors associated with the user. A digital processor matches the sensor data to a signature definition to determine whether the user is performing the activity. The signature definition is correlated to expected sensor data from each of the plurality of sensor and corresponding to the activity.

In another embodiment, a pod determines activity of a user. The pod includes a plurality of sensors capable of generating sensor data based upon sensed characteristics of the user. The pod also includes a memory that is capable of storing a signature definition based upon a known activity. The pod also includes a processor coupled with the memory and the plurality of sensors. A match routine, having machine readable instructions stored within the memory, when executed by the processor, is capable of matching the sensor data with the signature definition to determine the activity. A transceiver is capable of communicating the activity to an external device.

In another embodiment, a system determines when a user performs an activity. The system includes a first pod configured with the user and a server. The first pod having a sensor for generating sensor data indicative of characteristics of the user and a first transceiver for wirelessly transmitting the sensor data. The server includes a processor, a second transceiver for receiving the sensor data, a memory for storing a signature definition corresponding to the activity and the sensor, and an algorithm having machine readable instructions that, when executed by the processor, are capable of matching the sensor data to the signature definition to determine if the user is performing the activity.

### BRIEF DESCRIPTION OF THE FIGURES

FIG. 1 shows one exemplary system for monitoring activity of a user based upon signatures, in an embodiment.

FIG. 2 is a table illustrating exemplary states and direct truth measurements determined by the pod of FIG. 1, in an embodiment.

FIG. 3 shows exemplary detail of the signature of FIG. 1.

FIG. 4 shows exemplary signature calibration and data flow within the pod if FIG. 1, in an embodiment.

FIG. 5 is a table listing exemplary types of signature, in an embodiment.

FIG. 6 shows the pod of FIG. 1 with an exemplary learning module, in one embodiment

FIG. 7 shows one exemplary scenario illustrating matching of a pattern to a signature definition of a signature with an associated match score, in an embodiment.

FIG. 8 shows an alternative scenario wherein a first portion of a pattern is matched to a first signature definition of a first signature with an associated first match score and a second portion of the pattern is matched to a signature definition of a second signature with an associated second match score.

FIG. 9 shows the pod of FIG. 1 configured with an interface that is supported by one or more application programming interfaces (APIs), in an embodiment.

FIGS. 10 through 14 show exemplary use of pods for determining location within a building, in an embodiment.

FIG. 15 shows one exemplary map determined from a computer within a building entered by a pod, in an embodiment.

FIG. 16 shows one exemplary housing of the pod of FIG. 1, in an embodiment.

### DETAILED DESCRIPTION OF THE EMBODIMENTS

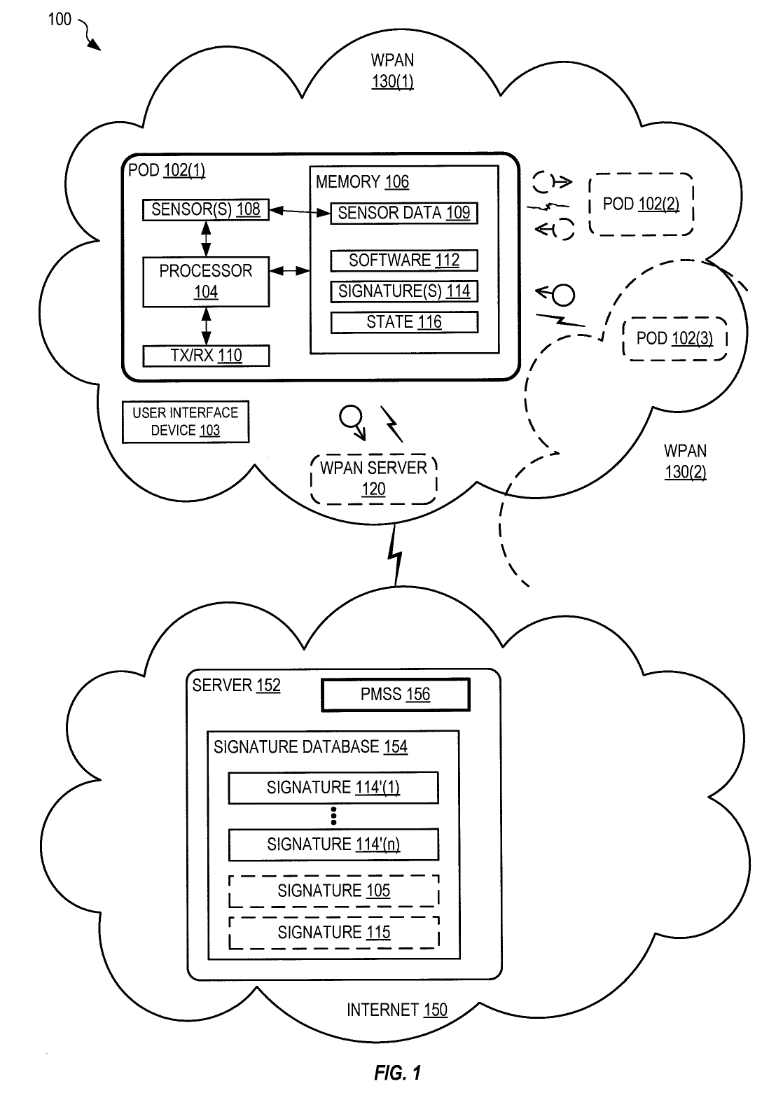

FIG. 1 shows one exemplary system 100 for monitoring activity of a user based upon signatures 114. System 100 is formed of one or more pods 102, an optional wireless personal area network (WPAN) server 120, and a pod management software system (PMSS) 156 configured within a server 152. The monitored activity is for example a class of activity such as one of running, walking, cycling, and so on.

Pod 102 is a computer that includes a processor 104, memory 106, one or more sensors 108 and a transceiver 110. Pod 102 includes, within memory 106, at least one signature 114 that defines expected signals from one or more sensors 108 for a particular activity of the user. Software 112 includes machine readable instructions, stored within memory 106, that when executed by processor 104 implement functionality of pod 102, as described in detail below. Software 112 includes algorithms that match activity sensed by sensors 108 to signatures 114 to change a state 116 that defines one or more of a location, a speed, and direction of the user. State 116 may define other determined and/or estimated states and activities of the user that are detectable by, and/or determinable from, information sensed by sensors 108 and information received via transceiver 110. Pod 102, when associated with a user, may be worn by the user or may be mounted on a device or vehicle used by the user. For example, pod 102 may be mounted on top of the user's shoe/boot, around the user's ankle, on the user's knee, at the user's waist, at the user's shoulder, on the user's head, and on one of the user's arms. In another example, pod 102 is mounted on one of a hockey stick, a rowing oar, a walker, a wheel chair, a tool belt, a bicycle, and an inline skate. Pod 102 may be mounted elsewhere without departing from the scope hereof. Different mounting techniques may be used for each pod based upon expected activity and application. For example, a pod may be mounted by one or more of: one or more straps that wrap around part of a user or equipment, hooks that mechanically couple with eyes configured in clothing and/or equipment; proprietary rail mechanisms that connect to a shoe mount; and a clamp mechanism for attaching to shoe laces.

Pod 102 communicates (e.g., wirelessly) with a user interface device 103 to interact with a user of pod 102. User interface device 103 may represent one or more of a smart phone, a desktop computer, a tablet, a notebook computer, a head-mounted activity display, and other similar devices. In one example of operation, pod 102 interacts with the user by communicating with user interface 103 to display activity and state information, and to receive control inputs from the user. User interface device 103 may provide one or more of visual outputs, audio outputs, and tangible outputs to the user. Similarly, user interface device 103 may receive one or more of visual inputs (e.g., gestures using a camera or other sensor), audio inputs (e.g., voice commands), and tangible inputs (e.g., button presses, taps, head movements, etc.). In certain embodiments, user interface device 103 may be combined and/or physically coupled with pod 102. In one embodiment, user interface device 103 is electrically coupled (e.g., wired) with pod 102. In one embodiment, user interface device 103 and pod 102 communicate using WPAN server 120. Other user interface devices may be used by pod 102 to interact with the user without departing from the scope hereof.

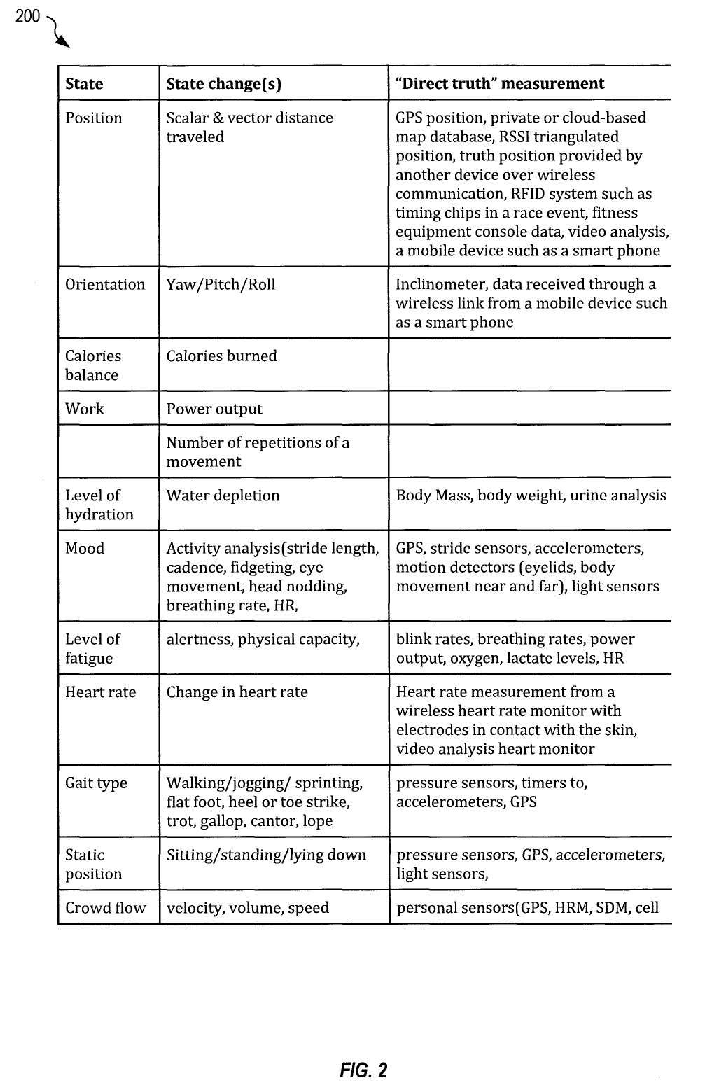

FIG. 2 is a table 200 illustrating exemplary states 116 determined by pod 102, where each state is shown with an associated direct truth measurement. These direct truth measurements represent trusted information that may be used within pod 102 for calibration purposes. Direct truth measurements may be sensed by sensor 108 within pod 102 and/or provided to pod 102 via transceiver 110. For example, pod 102 may receive direct truth measurements from one or more of WPAN server 120, other pods 102 (e.g., pod 102(1) may receive direct truth measurement data from one or both of pod 102(2) and pod 102(3)), and/or from PMSS 156. As described below, direct truth measurements may be used within pod 102 to automatically calibrate state changes indicated by signatures 114.

Pod 102 either (a) attaches to, is worn by, or is otherwise coupled with, the user, or (b) attaches to equipment or a vehicle used by the user. Pod 102 may also be attached to an animal or another object without departing from the scope hereof.

Pod 102 uses transceiver 110 to communicate with optional WPAN server 120 within a WPAN 130. WPAN 130 is for example a wireless network formed around the user that facilitates wireless communication between pods 102, WPAN server 120, and optionally other devices. WPAN 130 may be implemented using one or more wireless protocols selected from the group including: Bluetooth, Bluetooth Low Energy (BLE), ANT+, WirelessHART, Zigbee, RFID, Bluetooth 3.0, 802.11a/b/g/n, and GPRS/3G/LTE cellular data, or any other similar wireless communications protocol. That is, transceiver 110 implements one or more wireless protocols to facilitate communicate to and from pod 102.

Optional WPAN server 120 is for example a computer that wirelessly communicates with pods 102 within WPAN 130 and also wirelessly communicates with the Internet 150, for example by using Wi-Fi or other similar connectivity. WPAN server 120 thereby operates as a bridge between pods 102 and PMSS 156. In one embodiment, WPAN server 120 is one of a smart phone, a personal digital assistant, a tablet computer, a notebook computer, and other similar devices. In another embodiment, server 152 and WPAN server 120 are combined within the same device, wherein PMSS 156 communicates with pods 102 without using Internet 150. Where WPAN server 120 is not included, WPAN 130 may be formed by pod 102 (or another device) to facilitate communicate with other devices (e.g., external sensors) with wireless capability.

Pod 102(1) may also communicate with a pod 102(3) that is associated with a different user when that user is close enough to the user to enable communication between pod 102(1) and pod 102(3). That is, although pod 102(1) operates within WPAN 130(1), pod 102(1) may also communicate with a pod 102(3) operating in a separate WPAN 130(2). For example, pods 102(1) and 102(3) may exchange signatures 114 and other information for improved sensing of the users' activity.

Sensor 108 may represent one or more of the following: Accelerometer, Microphone/Noise threshold, Perspiration (e.g. Galvanic Skin Response), Compass, Temperature (e.g., Core Body/Ambient/Skin), Inclination, Gyroscope, Training Effect/Excess Post-exercise Oxygen Consumption, Altimeter, Short-range Radar/Sonar/Laser, Barometer, Camera, Ambient Light, Global Navigation Satellite System (GNSS), Electromyogram, Receiver Signal Strength Indication for one or more of the wireless protocols, Electroencephalogram, Respiration & VO₂, radio frequency identification (RFID) or other near field communication (NFC) (e.g. ChampionChip), and photoplethysmograph (PPG) heart rate. In one example of operation, software 112 controls sensors 108 to sense activity and other characteristics of the user and to match signals from these sensors to one or more signatures 114 that identify the actions of the user to update state 116 accordingly.

### Sensor Fusion

Information from sensors 108 may be combined (a fusion of sensors) to improve sensing of the user's activities. For example, measuring barometric pressure to determine altitude often yields an inaccurate result. Likewise, using GNSS alone to determine altitude also often yields an inaccurate result. However, a fusion of measurements from both sensors yields a result that is more accurate than is obtained when using either sensor on its own.

Accordingly, signatures 114 are configured to match information sensed by one or more sensors 108 to improve identification of user activity. For example, one signature 114 that determines when the user sits may utilize sensed information from sensors 108 including a GNSS, at least one accelerometer, and a gyroscope that in combination detect when the user stops moving, descends, and sits in a chair.

In one embodiment, raw data captured simultaneously from inertial and barometric sensors and information from GNSS receiver is collected and processed offline to identify characteristics and define signatures that make better use of the inertial and barometric sensor data for speed and elevation accuracy for use when GNSS data is not available, or when GNSS is disabled to reduce power consumption.

Pod 102(1) also includes software 112 that comprises machine readable instructions stored within the memory 106 and that are executed by the processor 104 to match collected sensor data from the one or more sensors 108 (with or without fusion) to signature 114. When the sensor data matches signature 114, software 112 determines a state and/or activity of the user based upon the matched signature 114. Software 112 implements a state management subsystem (see state manager 406 of FIG. 4) that transitions states 116 of the user, based upon matches of sensor data 109 to signature 114. States 116 may be validated periodically by one or more of (a) “direct truth” sensor measurements when available, (b) calculation, and (c) collaboration.

Sensor fusion may utilize one or more algorithms, located within software 112, that combine data from multiple sensors. For example, the algorithm may use one or more of a Kalman filter (including variants such as unscented Kalman filter (UKF)), least squares, a weighted average, and a particle filter.

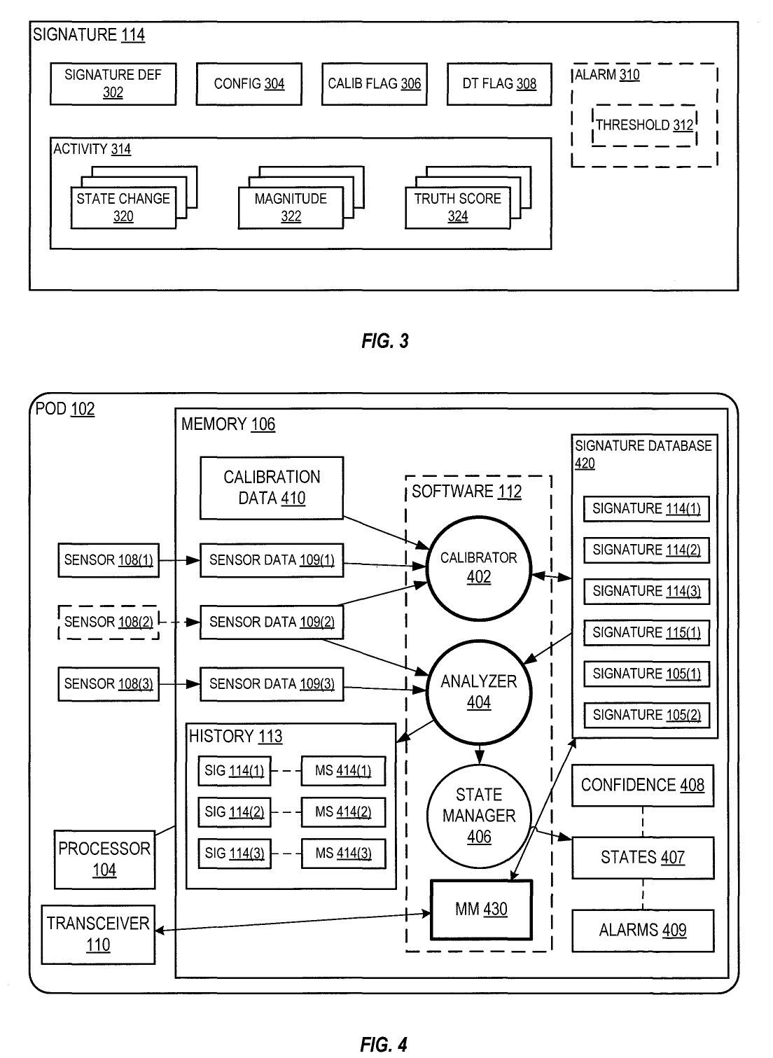

FIG. 3 shows signature 114 in further detail. Signature 114 includes a signature definition 302 that defines a pattern of sensor data (e.g., as received from one or more sensors 108) that is associated with an activity 314 of the user. In one example where pod 102 has three accelerometer sensors 108 that are positioned orthogonal to one another, signature definition 302 may define data patterns for two or more of these sensors based upon their orientation and the user activity being identified. Signature definition 302 may also reference one or more other signatures 114. For example, if a first signature is used to match a rotation of five degrees to the right, as detected by a gyroscope sensor, a signature definition 302 of a second signature may specify nine occurrences of the first signature for detecting a right turn of forty-five degrees. A signature definition 302 of a third signature may define two occurrences of the second signature to detect a right turn of ninety degrees.

Signature 114 also defines an activity 314 (state update information) that is based upon the sensed user activity matched by signature definition 302. Activity 314 defines at least one state change 320 that has an associated magnitude 322 and an associated truth score 324.

Optionally, signature 114 may also define an alarm 310 that has an associated threshold 312. Pod 102 may transmit an alarm message when threshold 312 repetitions of signature definition 302 are matched. In one example of operation, pod 102 is worn by an elderly person and configured with signature 114 that detects when the person falls. Threshold 312 is set to immediately trigger alarm 310 causing pod 102 to send an alert message via transceiver 110 to a remote wireless device with a capability to initiate a phone call, for example to 9-1-1. In another example, pod 102 is worn by a user and configured to detect repetitive motions, wherein threshold 312 is set to trigger alarm 310 when a certain number (set within threshold 312) of repetitions of a certain movement matched by signature definition 302 are detected, thereby alerting the user to interrupt the repetitive movement to avoid injury.

Truth score 324 represents the accuracy of magnitude 322 compared against a history of “direct truth” measurements. For example, where signature 114 uses data from accelerometer sensors 108 to detect a running speed of nine miles per hour (i.e., magnitude 322 is set to 9 miles per hour), truth score 324 of 90% indicates that magnitude 322 is determined 90% accurate for that state change 320.

Signature 114 includes configuration data 304 that identifies one or more sensors 108 from which data is used to match signature definition 302. For example, configuration data 304 may define a type, a location, and an orientation of one or more sensors 108 from which to match sensor data to signature definition 302. For example, configuration data 304 may define that signature definition 302 is for an accelerometer, located on a leg of the user and oriented vertically. Signature 114 may also include a calibration flag 306 that indicates whether signature 114 requires calibration. For example, if signature 114 is generic, calibration flag 306 would indicate that calibration for a particular user using pod 102 has not been performed. Signature 114 may also include a direct truth flag 308 that indicates whether signature 114 provides direct truth data. For example, where signature 114 is associated with a GNSS sensor 108, speed values from the GNSS may be used as direct truth values for calibration of other signatures.

FIG. 4 shows pod 102 in further detail, illustrating exemplary signature calibration and data flow. Pod 102 includes a calibrator 402, implemented as machine readable instructions that are stored within the memory and executed by processor 104, to calibrate signature 114 to the user of the pod. Pod 102 is shown with three sensors 108(1)-(3) that provide sensor data 109(1)-109(3) for signature matching and direct truth measurements. FIGS. 1, 3 and 4 are best viewed together with the following description.

Calibration data 410 includes parameters that define characteristics of a user of pod 102. For example calibration data 410 may include a user identifier (for when pod 102 is used by multiple users), and for each user may define one or more of: weight, height, age, gender, body composition, genetic composition, gravitational acceleration (e.g., to allow for gravitational variation based upon location on earth), and mounting position. Calibration data 410 may thereby be used to calculate a relative adjustment factor to magnitude 322 of certain signatures 114.

### Generic Signature Database

As shown in FIG. 1, a server 152 is accessible via the Internet 150 and contains a signature database 154. Signatures 114 within database 154 may be generic, in that they are configured for a user with generic characteristics. Each generic signature 114′ is associated with one or more sensors 108 and identifies an activity 314 when matched to sensor data 109. Sensor data 109 is stored within a buffer within memory 106. Generic signatures 114′ are loaded into pod 102 and configured for that user associated with the pod. For example, generic signature 114′(1) is loaded into pod 102, is characterized to the intended user of pod 102 based upon configuration data 304, and is then stored as signature 114 within pod 102.

Each pod 102 has a signature database 420 that stores a plurality of signatures 114 that are selected based upon expected activity of the intended user of pod 102. For example, where the user intends to use pod 102 while running, database 420 is loaded with signatures 114 that match running activities of the user.

Within each signature 114, activity 314 defines one or more corresponding state changes 320, each state change 320 having a magnitude 322 that may be assigned, learned, shared, and/or adjusted over time. For example, a signature 114 is associated with sensor 108 that is an accelerometer and that is attached to a user's foot. Signature definition 302 defines a pattern of acceleration and deceleration that spans 0.67 seconds, bounded at the start and finish by a vertical foot impact and the associated activity 314 defines first state changes 320 that defines a change in horizontal position with magnitude 322 of 40 cm, and a second state change 320 that defines a calorie burn with a magnitude 322 of 0.2. Thus, each match of sensor data 109 to signature definition 302 generates activity 314 indicating state changes 320 with magnitudes 322. A second signature 114 may also use sensor data 109 from the accelerometer to determine when the user's foot is at a specific height. Yet another signature 114 may use sensor data 109 from the accelerometer to determine when the user's stride is a specific length. Yet another signature 114 may use sensor data 109 from the accelerometer to determine when the user's cadence is at a defined rate. By including a combination of signatures 114 within pod 102 that utilize the accelerometer data, real-time analysis of the user's foot motion may be performed.

In a further example, where a signature 114 is associated with movement of a user's foot, signature definition 302 may include data representative of the foot striking the ground. This signature 114 may match a casual walking step, a brisk walking step, a light jogging step, an intense jogging step, a full sprint step, a single or multiple stair climb step, a stair descending step, a shuffle, and a skate stride. Other types of signature are not necessarily bounded by foot strikes and may match foot motion such as occurs with measuring a cycling cadence, movement on an elliptical exerciser, movement on a stepper exerciser, and movement on other low impact exercise equipment, movement during Nordic ski stride, movement in an elevator, and so on.

A plurality of signatures 114 may be defined to measure movement more accurately. For example, signatures for each of a plurality of different running speeds (e.g., 4 MPH, 5 MPH, 6 MPH, 7 MPH, 8 MPH, and 9 MPH) may be included within pod 102, wherein pod 102 may then determine the user's current running speed to within 1 MPH. The number of signatures 114 and spacing of magnitude of the detected activity between each signature may be selected for optimal performance of pod 102. Accuracy may be further improved by including multiple signatures for each speed, where different signatures for a particular speed are matches to different stride types (e.g., a difference in stride between starting out and when the user is tired).

In one example of operation, a discus thrower attaches pod 102 to his/her foot where foot movement is of particular interest to the thrower, since during a discus throw, the throwers foot performs both forward movement and changes in orientation. Where no existing signature matches this complex foot movement, pod 102 learns one or more new signatures (e.g., by matching portions of the movement to existing signatures and/or creating new signatures based upon the sensed movements). The newly created signatures may then be used to monitor the movements of the foot during repeated throws to collect specific performance information and provide an indication of consistency in repeated throws for example.

In another example, learned signatures may be shared with other users. For example, a golf professional creates a set of signatures 114 that match one or more aspects of a golf swing. By sharing these signatures with clients, the golf professional receives signatures logs from the clients' pods 102 that provide indications of how the clients' perform their golf swing. Similarly, each client may use pod 102 to create one or more signatures that match their golf swing and then share these signatures with the golf professional. The golf professional may then group these signatures into modalities to create conglomerate signatures matching each of the modes across the population of samples. These conglomerate signatures then allow the golf professional to classify the golf swing of a new client by attaching pod 102 (with these signatures installed) to the new client.

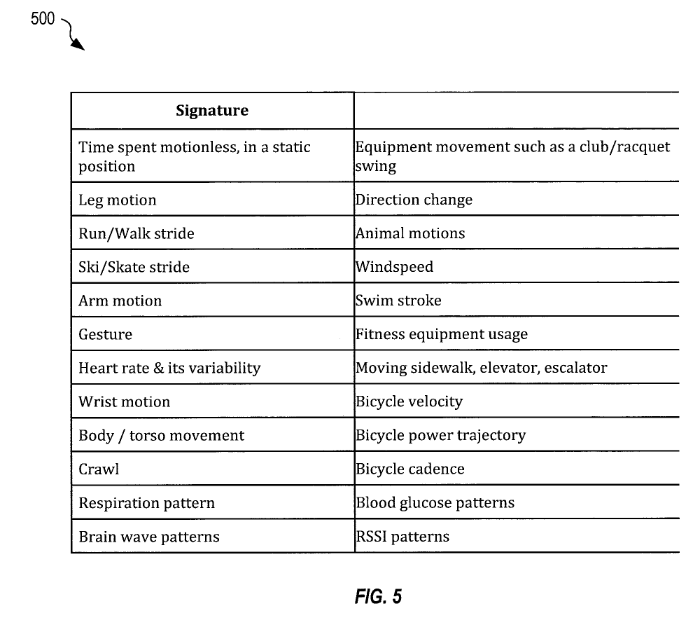

Exemplary types of signature 114 are listed in table 500 of FIG. 5.

### Signature Learning and Recognition

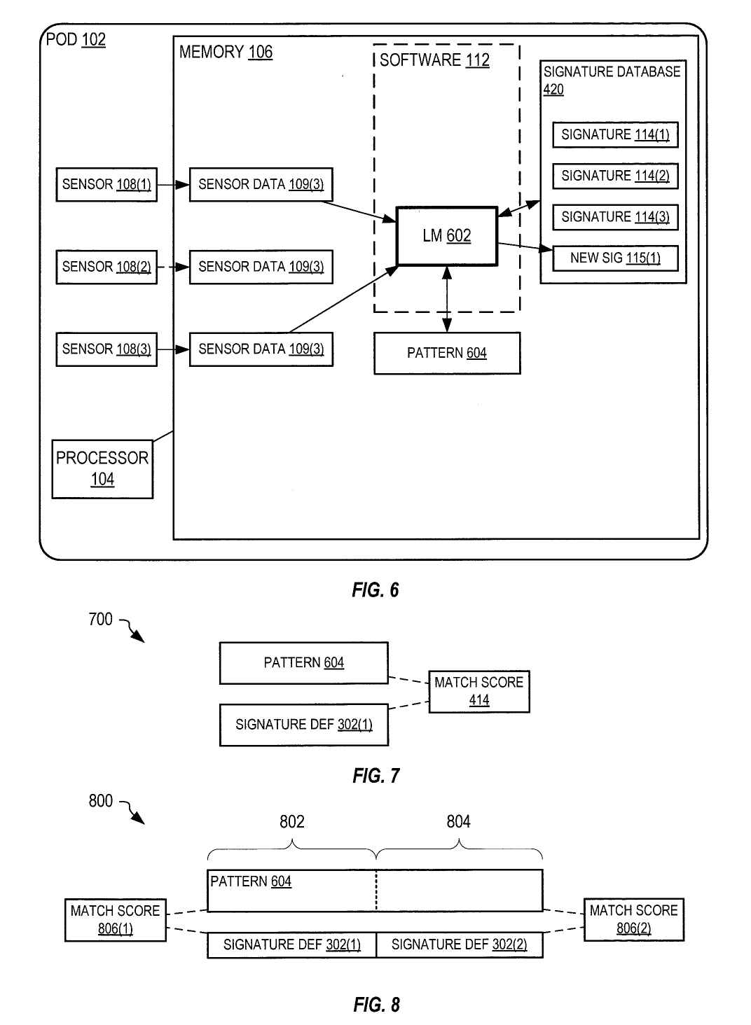

FIG. 6 depicts an exemplary pod 102 incorporating a learning module 602, in one embodiment. Learning module 602 is implemented as machine readable instructions stored within memory 106 and executable by processor 104 to identify a repetitive pattern 604 within sensor data 109 of one or more sensors. Learning module 602 is configured to process sensor data 109(1) and 109(3), buffered within memory 106, to identify repeats of pattern 604, such as a pattern of acceleration and deceleration bounded at start and finish by ground strikes where sensors 108(1) and 108(3) are accelerometers positioned proximate a foot of a user walking or running. In one embodiment, learning module 602 recognizes patterns 604 by matching a first portion of sensor data 109 with other portions of sensor data 109. Where learning module 602 is configured to find repeating patterns in multiple sensors 108, matching occurs synchronously across associated sensor data 109 from those sensors.

Once pattern 604 is determined, module 602 then searches signature database 420 to determine whether any portion of pattern 604 matches signature definition 302 of any existing signature 114. If no match between pattern 604 and signature 114 is found, then module 602 creates a new signature 115(1) and determines the state change 320 and magnitude 322 of one or more activities 314 associated with the new signature 115 based upon one or more of (a) sensor data 109 of other sensors 108 that provide “direct truths,” (b) information received through transceiver 110, and (c) or by solving for the resulting change in state from the occurrence of one or more unmatched signatures by combining with other existing signatures with known state change that occurred between two “direct truth” measurements of a given state. For example, an unmatched series of sensor measurements together with its measured state change may result in the creation of a new signature to match the measured state change. This may result from a series of sensor measurements partially matching a signature and partially not.

Where pattern 604 matches an existing signature definition 302, a match score (e.g., match score 414, FIG. 4) defines an accuracy level (e.g., a confidence level) of the match. With a match, the system will recognize the matched portion of the data buffer as a repetition of the particular signature with its match score. A match score is a measurement of how well a given set of sensor measurements matches with a given signature, and is used to determine the “best-fit” signature to the sensor measurements. The match score may be determined by correlation, and may use techniques borrowed from speech recognition, for example. Truth score (e.g., truth score 324) is a measurement of how well a given signature's state change agrees with direct truth measurements when available, and may be used to choose among multiple signatures that have similar match scores to a particular set of sensor measurements.

Where pattern 604 matches more than one existing signature 114, new signature 115 is defined as including each of the matched existing signatures 114 with an associated match score or confidence score. Where a first portion of pattern 604 matches a first signature 114, and there is no match for the remaining portion of pattern 604, this remaining portion is considered new. Software 112 may then define this remaining portion of pattern 604 as a new signature (e.g., new signature 115).

FIG. 7 shows one exemplary scenario 700 illustrating pattern 604 matched to signature definition 302(1) of signature 114(1) with a match score 414. Based upon scenario 700, signature definition 302 of new signature 115(1) identifies signature 114(1) and includes match score 414.

FIG. 8 shows an alternative scenario 800 wherein a first portion 802 of pattern 604 is matched to signature definition 302(1) of signature 114(1) with a match score 806(1) and a second portion 804 of pattern 604 matched to signature definition 302(2) of signature 114(2) with a match score 806(2). Based upon scenario 800, signature definition 302 of new signature 115(1) identifies signature 114(1) with match score 806(1) and identified signature 114(2) with match score 806(2).

### Signature Sharing

As illustrated in FIG. 4, within pod 102, a management module 430 may share one or more signatures 114 from signature database 420 with other devices (e.g., other pods 102 and server 152) connected to WPAN 130 and or Internet 150. For example, within pod 102(1), module 430 may share one or more signatures 114, including newly learned signature 115(1), with pod 102(2) via transceiver 110. Within server 152, PMSS 156 matches received signatures 114 against signatures 114′ stored within signature database 154, wherein the identity of similar signatures 114′ may be returned to pod 102(1) for possible inclusion within signature database 420 of pod 102(1). That is, pod 102(1) may automatically receive additional signatures 114 from server 152 that are relevant to its planned activity. This is particularly useful where pod 102(1) sends new signature 115(1) to server 152 and receives one or more similar signatures 114 in return.

For example, where pod 102(1) learns new signature 115(1) from sensor data 109 collected while attached to a user that is walking, PMSS 156 may automatically identify one or more signatures 114′ with similar signature definitions and wirelessly send these identified signatures back to pod 102(1). These identified signatures 114′ may represent signatures for identifying other walking strides in a variety of gait groups for example. Further, where PMSS 156 identifies best-matched signature 114′, pod 102(1), upon receiving signature 114′ from server 152, may update unknown parameters of new signature 115(1) based upon similarity of signature 114′. That is, where new signature 115(1) does not yet define one or both of state change 320 and magnitude 322, pod 102(1) may automatically “learn” initial values for one or both of state change 320 and magnitude 322 from best-matched signature 114′ prior to self-automatic calibration.

When a very similar stride exists in one or more gait groups, the PMSS identifies the best-matched gait group using a multiple signature comparison to each candidate gait group. Again, the PMSS shares other signatures from the best-matched gait group with the pod.

As shown in FIG. 1, pod 102(1) is in wireless communication with pod 102(2) and may share signatures 114. For example, pod 102(1) may send one or more signatures 114 to pod 102(2), wherein pod 102(2) may automatically accept or ignore each received signatures 114 based upon certain criteria being met (e.g. match/truth score above threshold). Pod 102(2) may accept and store zero, one or more of the received signatures 114′ within signature database 420 and modify a flag to indicate that the match/truth scores of the received signatures 114′ need to be re-calculated, e.g., based upon actual usage of pod 102(2). Signatures shared between pods 102 may be used to build aggregate signatures that better match a broader population (see state change calibration and adjustment, below). Signature sharing between pods 102 is managed using management module 430.

Calculating Changes to State from Signature Repetitions

Within pod 102, analyzer 404 matches one or more signatures 114 to sensor data 109 and cooperates with a state manager 406 to calculate any change in one or more states 407 of the user based upon the one or more matched signatures. State manager 406 may accumulate state changes from a series of matched signatures 114 to determine state 407, in a manner similar to dead reckoning.

In one example of operation, where multiple signatures 114 are matched to sensor data 109, state manager 406 may use selection criteria, based upon match scores 414 for each matched signature 114, which may also include calibration data 410, to select one of the matched signatures that is most appropriate to determine any change to one or more of states 407. When changing state 407, state manager 406 may also generates confidence 408 to indicate a confidence level for state 407. For example, state manager 406 may generate confidence 408 based upon history 113, and in particular using match scores 414 within history 113. State manager 406 may also exclude erroneous signature matches based upon history 113. For example, where matched signatures within history 113 for the last few minutes indicate a running state and then a swimming signature is matched, state manager 406 may ignore that matched swimming signatures, even where that signature has a high match score, and particularly where subsequent matched signatures indicate a running state. Similarly, where history 113 contains matched signatures 114 that indicate that the user of pod 102 is gradually slowing down, from 9 mph, to 8.5 mph, then state manager 406 may infer that the next signature may indicate a speed of 8 mph. Such inference and its use may be enabled by assigning probabilities to various state transitions within state manager 406 (e.g., using a Markov model). Other context information may be used to determine probability of state transitions. For example, access to the user's personal calendar (e.g., as stored on a smart phone) and/or training calendar (e.g., as created within Garmin Connect, Training Peaks, and other similar training web sites). By learning (e.g., from past events to predict future events, or from scheduled future events) when the user is likely to perform certain activities are performed, the probability of certain state transitions may be adjusted. For example, if the user usually has a lunch time cycle ride on Wednesdays, the probability of state transitions based upon cycling signatures may be increased during this period.

### Signature-Triggered Alarms

Each of the plurality of sates 407 managed by state manager 406 may have any number of alarms 409 associated with it, where for example each alarm 409 defines a range or threshold of that state that triggers the alarm condition. When state manager 406 determines that a direct truth or calculated value of state 407 triggers an alarm 409, an alarm message is sent out over one or more wired or wireless communication channels to an external device or party of the alarm condition and/or signaled to the user through one or more of multi-coloured LEDs, a vibrator motor, and an audio codec.

In one example of operation, pod 102 includes one or more accelerometer sensors 108 and is worn by a user exercising. Alarm 409 associated with a calorie burn state 407 is set for a desired calorie burn threshold and when pod 102 determines that the calorie burn state 407 exceeds the threshold of alarm 409, alarm 409 is triggered and pod 102 sends a message indicating that the calorie burn threshold has been met.

In another example of operation, pod 102 includes a hydration sensor 108 and is worn by a user exercising. An alarm 409 is set with a hydration threshold associated with the hydration state, such that when the user's hydration level drops below the hydration threshold, alarm 409 is triggered and pod 102 sends a message indicating that the user needs to re-hydrate their body because of lost hydration from their workout.

In another example of operation, pod 102 includes one or both of accelerometer sensors 108 and a GNSS sensor 108 and is worn by a user, is mounted to a walking assistance support (e.g., a walker or a cane), or is mounted to a manual or motor-driven vehicle (e.g., a scooter or a wheelchair). An alarm 409 within pod 102 may be configured to trigger when the user moves outside of a predefined area.

In another example of operation, pod 102 includes one or both of accelerometer sensors 108 and a GNSS sensor 108 and is coupled with one of a security badge, a visitor badge, and an identification tag, each of which may be worn by a user (e.g., an employee or a visitor). Pod 102 is configured to monitor the movement of the employee throughout an above-ground or underground secured or partially secured facility. An alarm 409 is triggered when the employee strays from their authorized work areas, whereupon pod 102 sends a message to a security system associated with the area. In a similar example, alarm 409 is defined as an area relative to another pod 102, wherein the alarm 409 is triggered when the distance between the pods 102 exceeds a defined threshold (e.g., when a visitor strays from their hosting employee). In a similar example, movement of the user throughout an underground mine, process plant, or warehouse is monitored and one or more alarms 409 are configured to trigged to indicate proximity of the user to a desired storage bay, exit paths, equipment, known hazards, safety stations, violations of a restraining order, etc.

In one example, pods 102 are each attached to a different member of personnel working in a secure facility to monitor movement of the personnel and to assist with authentication and facility security.

In another example, pod 102 is attached to an elderly person within an elder care facility and operates to track the location of the person (i.e., provide an alarm when the person leaves or attempts to leave the building without authorization) and the activity of the person (e.g., detecting when the person does something they shouldn't and/or the activity level in general of the person as an indication of health).

In another example of operation, each of a plurality of pods 102 is mounted to a rowing oar of a boat, wherein an alarm 409 in each pod 102 is configured to trigger when a rowing stroke of the oar exceeds a threshold of difference in rowing stride as compared to the majority of oars. That is, the alarm 409 is defined relative to a group measure. For example, each rower in a group of rowers may receive a diagnostic or corrective instruction based upon their performance relative to performance of the group and a whole.

In another example of operation, each of a plurality of pods 102 is mounted to players on a sports team, data from the units are used to recognize rule violations: (i) High sticking in hockey, where the position and orientation of the pod mounted to the hockey stick indicates that a high sticking violation has occurred. (ii) Off-side in hockey or soccer, or football, where the position of a pod worn by a particular player, relative to other players, indicates that an offside violation has occurred.

In another example of operation, when mounted to livestock, pod 102 may send an alarm when one animal strays farther than a maximum distance from the rest of the herd, or when the animal goes beyond a given boundary as established by the operator.

In another example of operation, when mounted to a miner, pod 102 may send an alarm when one miner strays farther than a maximum distance from the rest of the crew, or beyond a given boundary as established by the site manager

Pod 102 may be configured to operate as a wireless repeater for alarm messages sent by other pods 102, thereby extending the operable range of a short-range wireless communication protocol.

### Signature-Triggered Control

Using the signature analysis of an individual's movements using the sensor fusion algorithms, it is possible to create a unique motion topography of the individual to be used for motion sensing.

Unlike prior technologies that were based on proximity and biometric analysis for motion sensing which are consistent across multiple users but restricted by the “play area” and proximity to other users, the pod 102 provides a better solution for these primary reasons: (a) each user would generate a unique signature different from those of other users allowing him/her to use the motion sensing end application with their exact sensitivity and feel allowing for better performance, greater realism in the video game domain and dynamic adaptation of new gestures/movements as pod 102 analyzes new data from the user, and (b) using the built in ANT/BLE/BT sensors which will be used for data transmission between pod 102 and the end application transceiver, it would be unnecessary to require other hardware peripherals such as infrared or camera sensors to analyze movements as pod 102 performs such analysis. These external sensors may continue to be used for Biometric related applications if required.

Signatures may be used to detect certain gestures and movements of the user. For example, signatures may be defined to detect certain arm movements made by the user and used to control external devices. For example, matched signatures may be used for remote control of other devices and systems that are configured to response to messages from the transceiver 110 of the pod 102 (e.g., configured to communicate using the built-in ANT/BLE/BT wireless capabilities of pod 102).

As more BLE and ANT nodes enter the market, pod 102 may be loaded with custom gestures that allow the user to perform tasks remotely via the built in PAN wireless sensors. In one example, pod 102 is loaded with one or more (e.g., a set) signatures 114 that match certain arm movements, wherein the matching of these arm movements is used to control play (e.g., start, stop, volume) of a home entertainment device. In another example, pod 102 is loaded with signatures that match certain foot movements, wherein the matched signatures are used to control resistance of a bicycle training apparatus.

Use the built in PAN sensors via automated and gesture based methods to dynamically communicate with environmental PAN sensors in an intuitive, secure and robust manner. For example, pod 102 is configured with signatures 114 that match certain hand gestures and is worn on a finger of the hand. Matching of these signatures are used to control another device (e.g., a heart rate monitor worn on the chest, a heads up performance monitor, and a media player held in a purse or pocket) attached to or worn by the user and configured to operate with the user's PAN.

### State Change Calibration and Adjustment

When two direct truth measurements are separated only by a repetition of one newly-learned signature, the direct measured state change and the number of repetitions of the newly learned signature may be used to calculate and assign a state change for the newly-learned signature.

When two direct truth measurements are separated by a combination of existing and newly-learned signatures, simple algebraic equations involving the direct measured state change and the number of repetitions of each signature may be used to assign a state change to the newly-learned signature.

When two direct truth measurements are separated only by a repetition of an existing signature, the direct measured state change and the number of repetitions may be used to adjust (calibrate) the assigned state change for the newly-learned signature, and the associated truth score of the signature may also be adjusted accordingly.

When two direct truth measurements are separated by a combination of existing signatures, simple algebraic equations involving the direct measured state change and the number of repetitions of each signature may be used to adjust the state change of one or more of the existing signatures, and the truth score of the one or more signatures may also be adjusted accordingly.

A signature having either a multi-modal distribution or a large standard deviation may be separated into multiple signatures that better represent the various non-uniform categories of the signature. This separation may result in higher match and truth scores for each of the multiple signatures that are created to address the non-uniformity.

A collection of similar signatures, possibly with one or more similar calibration inputs and/or sensor values, may be aggregated into a single aggregate signature to better represent a broader number of users. This combination may result in reduced variance in match and truth scores across a large number of users.

### Individual Pod Data Analyses

Analysis of data (e.g., raw data, signatures, direct truth measurements) for a pod 102 may be performed by processor 104 within the pod or may be performed by PMSS 156 executing on server 152, which may be either private or cloud-based. For example, pod 102 may send sensor data 109 (with or without preprocessing within pod 102 to reduce the volume) to server 152 for processing by PMSS 156. Server 152 may receive raw data from a plurality of pods 102 physically coupled with the user, where PMSS 156 includes one or more signatures 114 that match the raw data from multiple pods 102.

The analysis performed on the data of pod 102 includes dividing the raw data into segments, and matching those segments to signatures 114 within signature database 420. Where no signatures are matched, the potential signature is “learned” by pod 102 by evaluating state changes deemed relevant to the signature. In one embodiment, a Fast Fourier Transform is used to identify periodicity in the raw data to facilitate division of the raw data into segments for matching with signatures. Other spectral techniques, including wavelets, may also be used. A heuristic method may be used to identify a likely subset of known signatures, which may then be convolved with the segments. Such a technique, if normalized, will output a “goodness” of fit which may be used to establish a level of confidence/trust in the identification of the matched signature. Each matched signature is then used to adjust the user's current state. For example, if a “sitting down” signature is matched, the state will transition from standing to sitting, and if a signature for a running step of two meters in length is matched, the state “total distance travelled” is incremented by two meters. In another simple example, signature 114 is configured to use one or more accelerometer sensors 108 to detect and count steps, thereby allowing pod 102 to operate as a simple pedometer. Automatic calibration of signatures 114, based upon received direct truth measurements for example, increases accuracy of distance determined from counted steps, as compared with regular pedometer devices.

When the state is incremented/changed a confidence level in new value of the state is determined based upon both the confidence level in the previous state value and the confidence in the identification of the signature.

### Constraining Possible State Space

Pod 102 may be configured with, or have access to, layout data for an area (e.g., a building) in which it operates, where the area constrains the possible movement possible of the pod. State changes estimated within pod 102 using matched signatures are validated against the layout data and if any state change is invalidated by the layout data (e.g. the state change indicates that the user has walked through a wall), the associated state changes and truth scores for the matched signatures may be adjusted (e.g., to adjust stride length for one or more signatures).

In one example, pods 102 are attached to players playing an indoor sport (e.g., one of football, hockey, etc.) and used to determine location of each player, map play formation and activity. The collected information may be transferred to a coaching station where the coach may view team and individual performances. For example, the coach may evaluate player locations relative to one another during play and thus deduce player interaction and opportunities therefore.

In another example, pods 102 are attached to staff and/or patients in a medical facility wherein location and activity of the staff and/or patients may be monitored automatically. In another example, pods 102 are attached to staff working at a facility with hazardous areas, wherein information from each pod 102 is automatically analyzed to determine when staff are approaching or have moved into the hazardous areas. In yet another example, pods 102 are attached to staff at a restaurant to monitor movement and activity of each member of staff to allow better planning and management.

### Multiple Pod Data Analyses

Pod 102 may share (e.g., wirelessly) state data with other pods where this data may be used to calculate group (collective) state values (e.g., circular error probable (CEP) and spherical error probable (SEP)) or root mean square position, group velocity, etc., which may in turn be shared (e.g., wirelessly) with connected pods 102.

Multiple pods 102 may be attached to the same user (or on equipment used by that user) to determine state data from different positions. For example, the user may attach pod 102(1) to the head, and pod 102(2) to one foot. In another example, each of a plurality of pods 102 is attached to a different user of a group of users. By sharing information, pods 102 may provide a more holistic picture of the state of the user or group of users, and may improve the quality of information that each pod provides. The latter possibility may be facilitated by taking the level of confidence of each user's determined state. For example, if user “A” has a higher level of confidence in their current position (either because they have additional sensors, or very regular readily identifiable signatures), and a user “B” has a low level of confidence in their determined position. If a distance sensor (ultrasonic/radar, RSSI, etc.) determines that user “B” is within a certain distance of user “A”, then the position of user “B” may be corrected and the confidence level of the position increased. Similar concepts may be applied to other states that are shared between multiple pods 102.

### Synchronizing Time Across Multiple Pods

Like calculating a position, GNSS pseudo-range measurements taken by each pod may be used to solve for an accurate time, within microseconds. For example, where one in a group of pods includes a GNSS receiver, it may determine both direct truth location and direct truth time values from the GNSS satellites. Pods without GNSS receivers may then determine an accurate time (and location) by communicating with the pod having the GNSS receiver based upon distance between the pods. Thus, each pod is able to periodically adjust its sense of time to synchronize with the highly-accurate GNSS satellite clocks.

### Multiple Users Each Wearing a Pod

In a team or competition environment, each of a plurality of pods 102 may be coupled with a different one of a plurality of players to simultaneously track each player. Data from pods 102 is relayed to a central station using transceiver 110. The central station is for example positioned at the side of the rink/athletic field/track/facility or accessed via a mobile device (computer tablet, phone) by a coach or trainer. A coach/trainer may then connect to the central station to access data for each athlete, and/or view collective data patterns of the group. For example, the coach/trainer may use an application to access the data and to assess movements of all the individuals, allowing the coach to track the movement and formation of players through drills or competition. Pods 102 thereby provide detailed positioning information to help inform tactical development. Athletes may be provided feedback through the application, and may be provided with new targets for the next training session or competition. Pods 102, when mounted on multiple individuals, also have the ability to communicate with one another, which may allow each pod to determine individual positioning with increased precision, as well as provide information on proximity of individuals. In a team environment this may provide feedback on tactical formation, and for an individual sport, the multiple pods 102 may be used to determine distance/time between individuals during a race or similar competitive event.

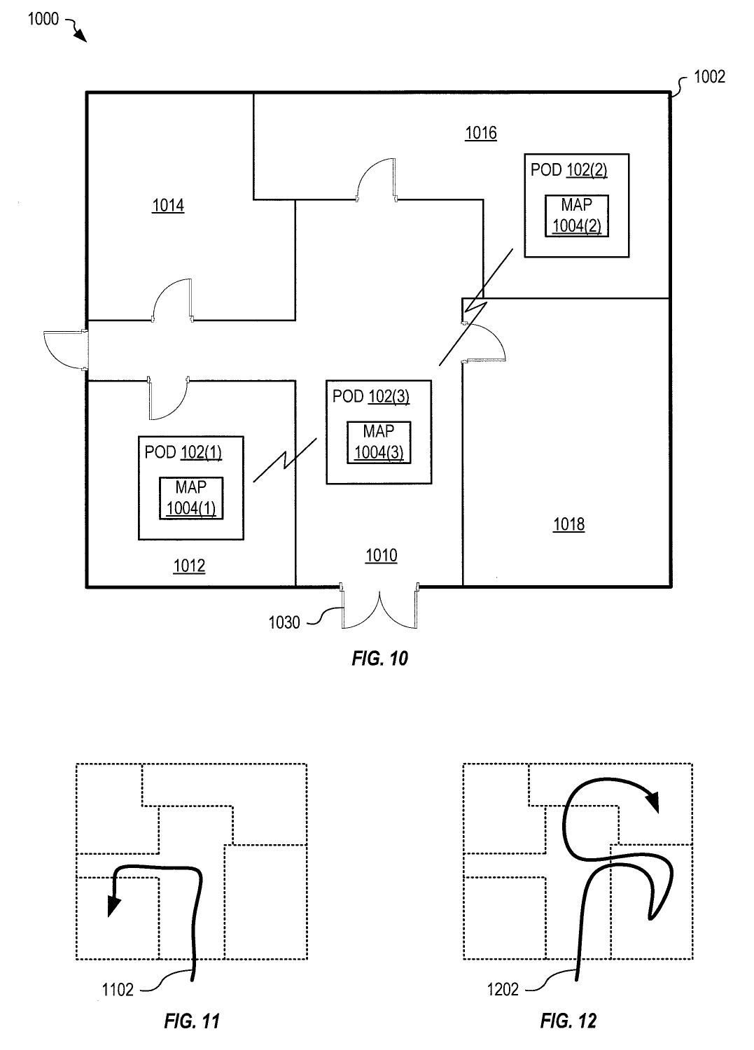

FIG. 10 shows one exemplary scenario 1000 where a first user wearing pod 102(1) has entered a building 1002 via a front door 1030 and moved to an office 1012 via a hallway 1010. A second user wearing pod 102(2) has entered building 1002 via front door 1030, visited an office 1018, and then moved via hallway 1010 to office 1016. Building 1002 has another room 1014 that is not visited by either the first or the second user.

Pod 102(1) creates, within memory 106 for example, a map 1004(1) of the first user's movements within building 1002 as shown in FIG. 11. In particular, map 1004(1) defines a path 1102 of the first user. Walls of building 1002 are shown in dashed outline within map 1004(1) of FIG. 11 for reference, but are not determined or stored within map 1004(1) by pod 102(1). Similarly, pod 102(2) creates, within memory 106 of pod 102(2) for example, a map 1004(2) of the second user's movements within building 1002, as shown in FIG. 12. In particular, map 1004(2) defines a path 1202 of the second user. Walls of building 1002 are shown in dashed outline within map 1004(2) of FIG. 12 for reference, but are not determined or stored within map 1004(2) by pod 102(1).

In scenario 1000, a third user wearing pod 102(3) enters building 1002 via front door 1030 and is within hallway 1010. Pod 102(3) detects presence of pods 102(1) and 102(2) and receives maps 1004(1) and 1004(2), respectively, therefrom to form map 1004(3) as shown in FIG. 13. Again, building 1002 is shown in dashed line within FIG. 13 for reference and is not stored within map 1004(3) by pod 102(3).

Since users (i.e., people) navigate stairs, doorways and corners differently, pod 102 may combine maps 1004 from multiple pods to improve accuracy. Maps 1004 are used by pod 102 to help constrain the solution space for performing calibration or calculating a state change. For example, map 1004 identifies the location of doorways and hallways and may be used when calculating state changes within pod 102 by excluding any state change that would violate physical possibilities, such as walking through a wall. Maps 1004 may be shared from pod to pod (e.g., shared between pods 102(1), 102(2), and 102(3)) through wireless transmission and may be used to specify in detail permissive zones for geo-fencing type applications or other signature based control applications that take location into account.

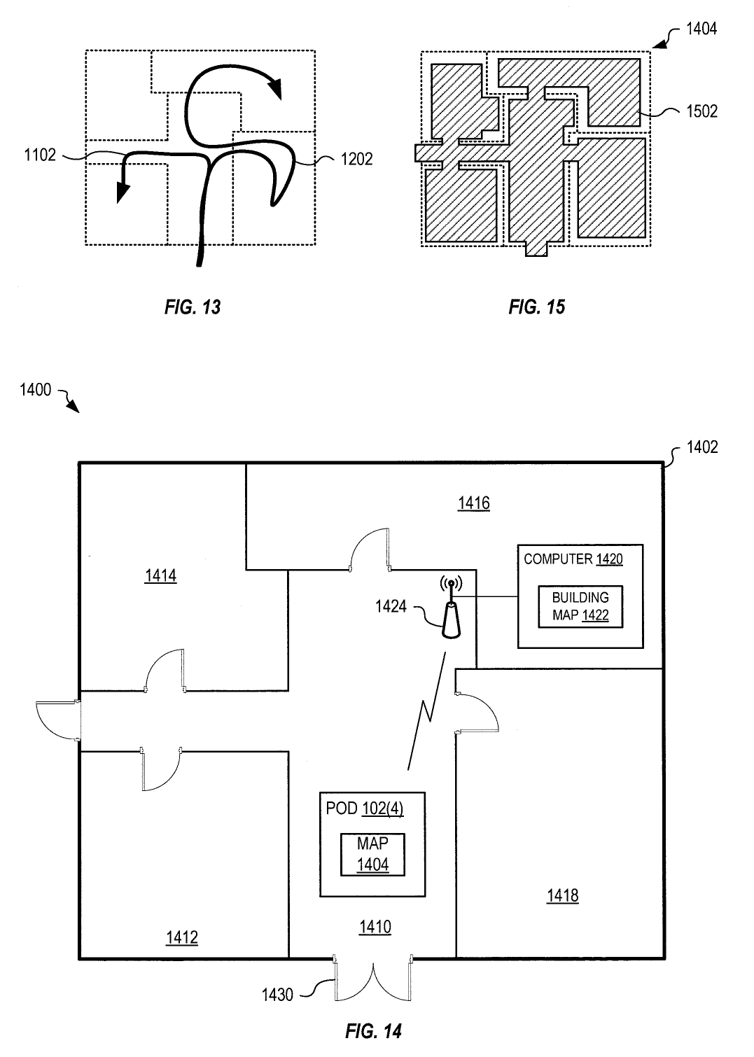

FIG. 14 shows another exemplary scenario 1400 where a first user wearing pod 102(4) has entered a building 1402 via a front door 1430. Building 1402 has a computer 1420 that includes a building map 1422 and is coupled with a wireless hotspot 1424. When within range of hotspot 1424, pod 102(4) requests building map 1422 (or at least part thereof) from computer 1420. As shown, pod 102(4) utilizes information received from computer 1420 to construct map 1404, which it then uses to qualify determined navigation within building 1402. For example, building map 1422 may identify freely navigable space (e.g., rooms 1412, 1414, 1416, and 1418, and corridor 1410) within building 1402, or may identify non-navigable space within building 1402. FIG. 15 is a schematic illustrating one exemplary map 1404 determined from building map 1422 of computer 1420 by pod 102(4). Map 1404 indicates a navigable area 1502 of building 1402 and uses that information to validate motion (e.g. based upon accelerometers) of pod 102(4) within building 1402. For example, if accelerometer based determination of movement indicates departure of pod 102(4) from navigable area 1502, algorithms operable within pod 102(4) may determine a most likely location of pod 102(4) based upon boundaries of navigable area 1502. Pods 102 may communicate with one another, when within communication range, to share location information.

Continuing with the example of FIG. 10, user of pod 102(3) has arrived slightly late for a meeting with a user of pod 102(1) within building 1002. The user of pod 102(3) has not been to building 1002 before, but based upon map 1004(3) and location information received from pod 102(1), the user of pod 102(3) may be directed to follow the path 1102 taken by pod 102(1), thereby finding the user of pod 102(1) within office 1012. In another example, coordinates of each pod 102 are queried through a peer-to-peer network or through an installed communication system (e.g., building Wi-Fi network) to locate each pod 102 and user thereof.

Pods 102 may also communicate other information to facilitate location of the pod. For example, ad-hoc networks of nodes may be built and used to transmit signature data collected by a first pod 102 to one or more other pods and/or computer systems for recognition. That is, where collected signature data is not matched by the first pod, it may be sent to a second pod 102 or system (e.g., computer 1420 of building 1402) where it is matched to a particular signature. For example, where the first pod 102 has not yet collected and constructed mapping data of a building (e.g. building 1402), but has captured an image of a yellow fire extinguisher, by sending that image to computer 1420, computer 1420 matches the image to one of more images of a known location within building 1402, and automatically sends the location information of these matched images to the first pod 102. Thus, even without direct location information (e.g., GNSS location data), the first pod 102 determines its location by communicating captured signature data (e.g., an image) for matching to signatures stored on other devices (i.e., other pods and/or computers) and thereby receiving location information in return.

Similarly, proximity to known fixed location in a building may allow a device to determine its location by recognizing signatures of the fixed location. Pods 102 may also communicate their locations to one another to determine distance therebetween. Where precise locations are not known, distance between devices may be determine through other means, such as RSSI.

Although the examples of FIGS. 10-15 are two dimensional, pod 102 may operate in three dimension and include movement on stairs, within elevators, and on other floors of a building without departing from the scope hereof.

Pods 102 communicate with one another, when within communication range, to share truth measurements for calibration and to reduce error in commonly determined data (e.g., location). For example, where two users, each wearing at least one pod 102, are performing an activity together, (e.g., running or cycling), pods 102 may communicate a determined travel distance such that accuracy may be improved.

### Multiple Pods on One User

A user may utilize multiple pods 102 (e.g., worn or on a vehicle ridden by the user) for recognizing and logging signature data. Each pod 102 records information (signature log) of the matched signatures 114, and assigns a timestamp to indicate when each signature was matched. At least part of the signature log may be wirelessly shared with PMSS 156, wherein the signature logs collected from multiple pods 102 are aggregate and matched to group (multi-pod) signatures 105 stored within signature database 154 of server 152 and signature database 420 of pod 102 that define complex body movements and associated state changes. Group signatures 105 may be used either to trigger a notification, and/or to determine one or more state changes. In one example of operation, PMSS 156 receives signature logs from a plurality of pods 102 and aggregates the identified state changes over time for comparison against one or more models of ideal body movement forms to determine an overall movement score. PMSS 156 may then advise on improvements to the detected form by suggesting adjustments to timing of body part movements, body angles, etc.

Where two or more pods 102 are attached to a user, the signature log of one of the pods may be shared in real-time with another of the pods. Within each pod 102, each matched signature is time-stamped and shared with other pods 102. A receiving pod 102 may aggregate both detected and received signature logs in real-time to match the signature logs against one or more group signatures 105 to identify complex body movements. Group signatures 105 may be used to trigger an alarm, control a device, and/or determine one or more state changes.

In one example, a user training for Nordic skiing wears multiple pods 102 that are positioned at different points on the body. The pods 102 cooperate to recognize the movement patterns and overall body alignment throughout the training. PMSS 156 processes matched signatures 114, 105 to assess the efficacy of the user's movement and form during the training, and may make suggestions on how the user's form may be improved.

In another example, a user wears multiple pods 102 during a dynamic fit to a piece of sporting equipment, such as a bicycle for example. Pods 102 are positioned at multiple points on the user's body to match signatures 114, 105 of expected movement patterns and body alignment throughout the dynamic fitting. PMSS 156 is then used to process matched signatures 114, 105 to assess and report on the efficacy of the user's movement and form. PMSS 156 may also make suggestions on how the user's form may be improved, and specifically, make suggestions to adjust the fit of the equipment.

In another example, a user wears (e.g., on one/both feet, knee, hip, on one/both wrists, bicep, neck, etc.) multiple pods 102 while running to match sensed movements to signatures 114, 105. PMSS 156 processes matched signatures 114, 105 to assess correctness of posture, stride type, overall gait efficiency, and to estimate power consumed by leg (e.g., foot strikes) and arm motion. Estimated power may be compared to calculate coarse power consumption expected for the activity, based upon body weight, vector distance, and time.

In another example, multiple pods 102 are worn (on one/both ankles, knees, hips, on one/both wrists, biceps, neck, etc.) by a user while swimming to match signatures 114, 105 of expected movement patterns and body alignment. PMSS 156 is then used to assess correctness of form, stroke type, overall stroke efficiency, and estimating power consumed by leg and arm motion of the user. Estimated power may be compared to calculate coarse power consumption expected for the activity, based upon body weight, vector distance, and time.

### Mounting

Pods 102 may be mounted anywhere on top of shoe/boot, around ankle, on the knee, waist, shoulder, arms, on a piece of equipment such as a hockey stick or a rowing oar, walker, wheel chair, tool belt, bicycle, in line skate, dogsled, etc. using some sort of a mount selected from the group including: hook into eyehooks, use a rail system to connect device to shoe mount, clamp into laces, within a Velcro arm/wristband, sewn or molded into an outer garment such as a ski suit, wetsuit, etc., and clipped onto the body.

### Sport Signatures

Pod 102 may be used for athletic sport analysis for both team environments and for individual use. Pod 102 is configured with one or more signatures 114 associated with the sport (team or individual) being played. For baseball for example, batting signatures may be generated from one or more pods 102 attached to a user and/or equipment of the user, and a comparison application may be used to compare the user's (e.g., an athlete) swing with signatures of an “ideal” swing. Similarly, pitching signatures may be detected for a pitcher and compared to signatures of other pitchers.

Similar techniques may be applied to other sports, wherein signatures may be detected for a racquet and/or a club swing, and may then be used in a simulator. E.g., a golf simulator may use detected swing signatures of a player to simulate movement of the player on a display screen.

Short term training may advantageously use short term signatures (e.g., acceleration signatures detected as an athlete starts moving from a stopped position) to perfect an athlete's initial burst of acceleration (e.g., at a start of a race), which is a very important aspect of sprint training. Pods 102 may also be used to count repetitions during anaerobic weight training, for example.

### Team Setting

Sensor fusion algorithms are used to analyze a group of similarly specialized athletes, such as pitchers in a baseball team, to generate one or more signatures 114, 105, 115 for detecting and comparing sport motions. Pod 102 may automatically identify an “ideal” sport motion from data collected from the group of athletes. In one embodiment, pod 102 identifies repeated sensed movements that are similar as a control sample and generates the ideal movements based upon those sensed movements. In another embodiment, a coach identifies a group of sensed movements as a control sample, wherein pod 102 uses those movements to determine the ideal sport motion. Thereafter, one or more pods 102 may be configured with signatures based upon the ideal motion such that motions (e.g., subsequent motions by the same or other athletes) may be compared to the ideal sport motion to show where improvement may be made.

One or more WPAN sensors may be used (e.g., in external peripherals such as a bat and ball) in conjunction with pod 102 to provide additional data, such as ball speed or swing speed/power for example. In one example, this external information, along matched signature information of the team, may be used to determine a collective signature of the team or may be used to differentiated one team member from the rest of the team. Thus, analysis of motion and acquisition of other data (e.g., ball speed, swing rate, etc.) by pod 102 may be used to generate a comprehensive signature of one athlete or of a team of athletes.

### Player Setting

Pod 102 is configured with one or more signatures 114, 105 for ideal sport motion using one or more sensors 108. For example, a coach may implement signatures 114, 105 that match ideal or desired motions for one athlete, wherein one or more pods 102, configured with these signatures are attached to the athlete. Pods 102 may then be used to collect information of the athlete's movement and determine when that movement conforms to the ideal sport movement. This configuration facilitates analysis for one-on-one training, ideally between the coach and the athlete. The athlete may use one or more pods 102 configured with signatures 114, 105 when training towards achieving the ideal sport movement. Alternatively, one or more of signatures 114, 105 may be pre-programmed into pod 102 such that the athlete, when performing to match these signatures, attains performance goals.

Pods 102 may also be used to determine one or more unique signatures 114, 105 of a movement by an athlete wearing the pods. The determined signature 114, 105, allows the athlete, or a coach of the athlete, to gain useful insight into the various advantages of the movement or to identify any flaws in the attributes of the movement. This may be advantageous for drafting and scouting in professional sports. Signatures 114, 105 may be shared wirelessly and will be described in more details in the following section.

### Application Programming Interface

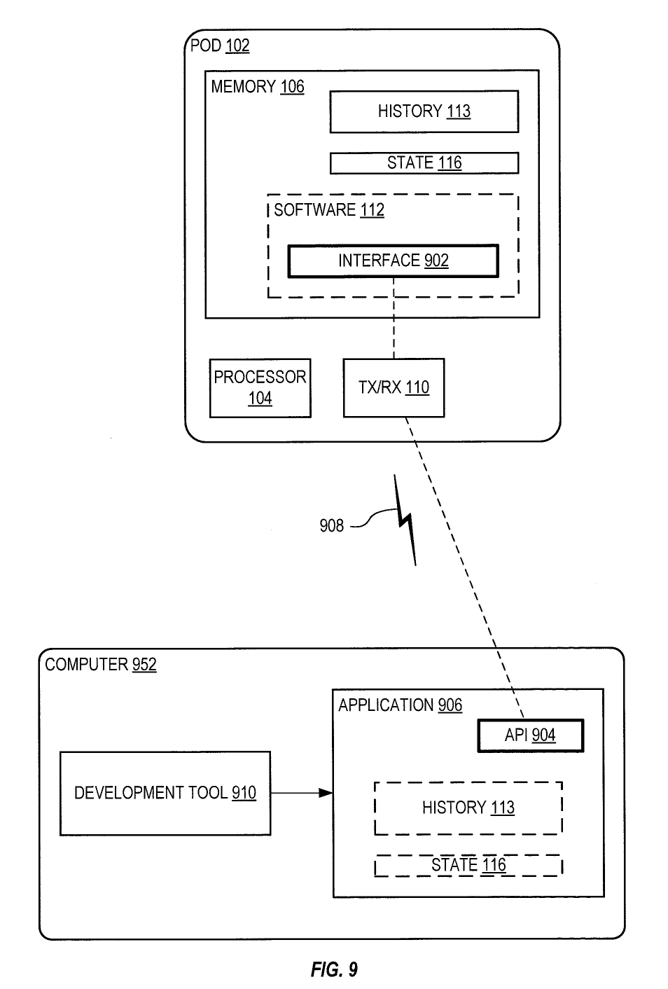

FIG. 9 shows pod 102 of FIG. 1 configured with an interface 902 that facilitates communication between pod 102 and other applications that utilize an application programming interface (API) 904. That is, API 904 facilitates development of applications (e.g., desktop applications, smart phone apps, embedded applications, and so on) that communicate with pod 102 via interface 902. For example, an application developer uses a development tool 910 running on a computer 952 to create an application 906 that communicates, using API 904, with pod 102, wherein other software within application 906 processes data retrieved from pod 102. For example, as shown in FIG. 9, application 906 may periodically retrieve history 113 and/or state 116 from pod 102 to determine specific information of the wearer/user of pod 102. In another example, application 906 is developed to collect, process, and display signatures and associated data from pod 102. In another example, application 906 is embedded in other equipment, such as Ball/Bat/Golfing apparatus, and thereby communicated with pod 102 of a user of that equipment. API 904 may also facilitate development of simulators such as Golf, Batting and Basketball simulators, within the virtual sporting realm, that base simulations on determined movements and states matched by signatures retrieved from one or more pods 102.

As shown, the primary interface to pod 102 is through wireless link 908. Interface 902 implements at least one protocol that may include multiple levels of control and communication. For example, a first level may be privileged and usable only by a manufacturer of pod 102. A second level may be open for sharing information between pods 102 and PMSS 156. A third level may be used for communicating with the user through a computer or mobile compute device application. Interface 902 may implement other levels and/or protocols that facilitate communication with other devices and/or at other priority/privilege levels.

### Short Term Training

Interval training, and other types of burst training, is one of the most important training techniques that is used in various individual sports such as Running, Biking, Racing, etc. In interval training extensive analysis may be performed on short-term, yet critical, portions of the training. For example, the initial burst of acceleration at the start of a sprint race is critical for perfecting the athlete's start to a race and improving overall time. Pod 102 may be used to generate and analyse the athlete's acceleration signature and aid in training the athlete for a perfect start to a race. This technique may also apply in cycling and running whereby the athlete must have the perfect form and acceleration zones to compete efficiently and effectively.

### Embodiments

### Strides

Pod 102 implements automatic periodic calibration of stride length and foot height data. In one example of operation, calibrator 402 sums the number of strides (e.g., accelerometer and gyroscope measurements matched to one or more signatures 114, 105) between two determined or known locations. Calibrator 402 may then calibrate used signatures based upon the distance between the two locations. For example, the two locations may be determined from GNSS measurements, wherein calibrator 402 may determine therefrom the movement for each used signature. Specifically, the distance is divided by the number of strides. In this manner, a series of accelerometer & gyroscope measurements within pod 102 are matched to one or more signatures 114, 105 where the state change for each matched signatures is specifically calibrated to the athlete using the pod(s). Calibrator 402 automatically and periodically calibrates signatures 114, 105 to ensure accurate modeling of each stored signature.

### Intelligent Positioning

Pod 102 may include a temperature sensor for measuring ambient temperature such that pod 102 may determine whether it is located indoors or outdoors. Indoor environments are typically within a few degrees of a nominal room temperature (e.g., 70F). When pod 102 is indoors, GNSS signals are less likely to be received however other wireless signals (e.g., wireless network access points and cellular base stations) may still be used for RSSI triangulation to determine location. Temperature measurement may be used in combination with GNSS signal strength measurements to provide additional evidence as to whether pod 102 is inside or outside.

Pod 102 may also be “context aware” wherein the history of matched signatures and determined locations may indicate, or provide additional determination of, a current location. For example, where the user has walked from outside a building to inside the building, by knowing the location when outside the building (e.g., using GNSS), the building the user has entered may be determined. In another example, pod 102 may determine its current context using wireless signals. For example, if a particular wireless signal is known to be at a certain location within a certain building, pod 102 increases knowledge of its current location when it detects that particular wireless signal. In another example, based upon detected forward and backward accelerations, average speed, etc., pod 102 may determine that it is in a vehicle. Other movement characteristics (e.g., sideways accelerations) may allow pod 102 to determine the type of vehicle (e.g., car, bus, train). Pod 102 may use maps and pattern recognition to determine a current location within a building as the user walks through the building. Pod 102 may also create its own maps of an unknown building based upon determined movements of the user within the building. Other information providing context to pod 102 may be used to determine a probability of whether the user is inside or outside. For example, weather information for the current location of pod 102 may be used to compare temperature of pod 102. Similarly, a current outside temperature may indicate whether the user is likely to be inside or outside.

Where images from a camera are available to pod 102 (e.g., via transceiver 110 from WPAN server 120 or from other devices), pod 102 may use captured imaged to determine or refine an estimated location. For example, images of doorways being passed through may enable pod 102 to recalibrate its estimated location (determined from other signatures that match walking, turning, etc.) with an identified feature of known location within the images. Pod 102 may use image recognition to identify specific features of known location within the image by matching at least part of the image to a street view or other similar visual database such that the location of pod 102 is learned, wherein pod 102 may then use that information for calibration of one or more determined states. That is, matching of the image to determine the location of pod 102 provides pod 102 with a direct truth measurement.

### Stairs

Pod 102 may be configured with (or may learn) one or more signatures 114, 105 for matching a user's movement when ascending or descending one or more stairs. For example, one signature 114 may match detected movement of the user ascending stairs and a second signature 114 may match movement of the user descending the stairs. Signatures 114, 105 may thereby identify when the user traverses the stairs, and in which direction. Once stored, the signature may be matched to indicate subsequent stairs traversed. Knowledge of location-based building conventions, or an actual building plan, may be used to calibrate these signatures, and may thereby also be used to determine location of the user within the building.

### Elevators

Pod 102 may use a similar approach to learn signatures 114, 105 for ascending or descending one or more storeys using an elevator. Pod 102 may be configured with one or more signatures 114 that match detected acceleration when ascending in an elevator, and one or more signatures 114, 105 that match detected acceleration when descending in an elevator. In one example, each of a plurality of signatures 114 may match a particular number of floors traversed by the elevator. Knowledge of location-based building conventions, an actual building plan, or GNSS measurements, may allow pod 102 to determine the actual number of floors traversed for calibration of the signatures.

### Escalators and Moving Sidewalks

Pod 102 may compensate for the constant directed motion of a moving sidewalk or escalator by removing its measured effect prior to signature analysis. Further, the detected presence of a constant motion may be used to differentiate between the shorter height stair step and the taller height escalator step when pod 102 is learning these signatures. Knowledge of location-based building conventions, an actual building plan, or GNSS measurements, may allow calibrator 402 of pod 102 to periodically calibrate these signatures.

### Data Logging

Pod 102 maintains a history 113 of signature matches, signature adjustments and signature calibrations. For example, as analyzer 404 matches one or more signatures 114, these signatures (or an ID thereof) are stored within history 113 in association with a match score 414. As shown in FIG. 4, signature 114(1) has a match score 414(1), signature 114(2) has a match score 414(2), and signature 114(3) has a match score 414(3). Match scores 414 indicate the confidence in the matching of sensor data 109 to each signature 114. History 113 may be shared with other pods 102, and other devices such as WPAN server 120, server 152, and other computers, smart phone, tablet, etc., configured to communicate with pod 102.

### Performance Degradation, Fatigue, or Mood Change

When pod 102 determines that the user is active for an extended period of time, pod 102 history 113 contains any adjustments made during that period to states change 320 of signatures 114, 105 within pod 102. For example, where calibrator 402 has changed state change 320 of signature 114 during a calibration process (e.g., adjusting one or more of the magnitude of stride length, foot height, and distance traveled, etc.), where pod 102 detects one or more of changes to stride length, pace, cadence, flight time, contact time, and joint angle, and other changes to crania-caudal and other movements in the medio-lateral axis over successive strides, it may send a message to alert the user (or an attendant/caregiver, coach) using interface 902.

For other activities, such as when monitoring arm or head movement, changes in the mood of the user may be determined from changes in the speed, frequency, jerkiness, etc. of such movements.

### Performance Improvement and Rehabilitation

When pod 102 determines that the user is walking, history 113 within pod 102 contains calibration adjustments to one or more signatures 114, 105. Where such adjustments detect an increase in one or more of stride length & height, and distance traveled per cadence, pod 102 may send a message via interface 902 to notify the user (or an attendant/caregiver, coach) of the improvement. This notification may be particularly useful when taking part in an at-home physical rehabilitation program. For example, signatures 114 may be defined for detecting different stages in rehabilitation, thereby providing indications of progress by the user.

In another example, a health professional may assign a range of exercises for a patient to complete during rehabilitation from an injury or surgery. Each exercise has an ideal motion when completed properly. Pod 102 is configured with one or more signatures 114, 105 that allow repetitions of properly performed exercises to be counted and time-stamped. Pod 102 may also be configured with signatures 114, 105 that identify and record improper movement. The patient takes pod 102 home and wears the pod when performing the exercises. When returning to the health professional, data may be downloaded from pod 102 into PMSS 156. Alternatively, the data may be uploaded to PMSS 156 from other locations (e.g., the patient's home) via the cloud. Thus, the health professional is able to monitor the patient's rehabilitation remotely, determine whether the patient is performing the assigned exercises correctly, and provide additional guidance and feedback based upon the data.

In one example of operation, pod 102 is used to record a patient's gait (or other movement) both before and after an intervention (e.g., surgery, Botox, orthodontics, medications, etc.) to track both the rehabilitation progress and overall improvement (or lack thereof) of both physical and emotional effects of the intervention.

### Sports Teams

Athletes on a sports team may each wear one or more pods 102 during training or during competition to collect a variety of statistical data. For example, each pod 102 may determine one or more of: distance traveled by the athlete during the game, the average pace and maximum speed of the athlete, a map of the area covered by the athlete on the playing area, heart rate changes over the game or training session, and, using data from all the athletes on a team, the player's proximity to other players. Based upon data collects for each athlete, the formation of the athletes during the game or training session may be determined. For competitive sports applications, pod 102 would be worn by the athlete in a location that would not interfere with play and would not injure other players in the event of contact. For example, for sports similar to and including soccer, pod 102 could be configured into the sole of the shoe or into the shin pad worn by the athlete.

Furthermore, one or more additional pods 102 could be configured with sporting equipment (e.g., a ball in soccer) that is used during a sporting event, where the additional pods 102 communicate with pods 102 attached to the athletes such that additional information is acquire. In one embodiment, pods 102 located within the equipment may utilize fewer sensors, since it could rely on a wireless protocol to pair with athletes' pods, and would trigger a sport-specific signature when detected by the athlete's pods. The additional pods could for example pair with the closest pod 102 (attached to an athlete) during competition to determine one or more of: possession statistics, athlete's time on the ball, percentage pass completion, possession changes/steals, and the last contact before the ball goes out of bounds or a goal is scored. With additional sensors, pod 102 within a ball could also determine the area covered by the ball during the game, as well as the acceleration, speed, height, and rotation of the ball when kicked.

The data from each athlete's pods 102 and sporting equipment's pods 102 may be relayed in real-time to coaching staff and referees on the sidelines, who may use the data for determining strategic and tactical support, for determining possession, and for determining rule violations, for example. Data from pods 102 may also be used by training staff during practice sessions and workouts, and by broadcasters that regularly use statistics during game analysis.

### Heart Rate Estimator

In one embodiment, pod 102 estimates a user's heart rate based upon determined activity of the user. Pod 102 learns one or more signatures for determining heart rates during various types of activity and inactivity, and may operate to estimate change to the user's heart rate. Calibration may be determined from actual heart rate measurements by the user. For example, the user may wearing a wireless heart rate monitor (often called a “heart strap”) with either electrodes or PPG placed against the skin for direct truth measurements that may be used to calibrate signatures within pod 102. In another example, pod 102 wirelessly couples with the heart strap for self-calibration. When the heart strap is not worn by the user, pod 102 estimates heart rate based upon the signatures and state management. This estimation approach is useful for a certain class of fitness participant, where the user's fitness level is not expected to change significantly on a day-to-day basis, and where the requirement of having to use a heart strap with each and every workout is considered inconvenient.

### Fatigue Monitor

Pod 102 may wirelessly couple with a heart rate monitor (heart strap) worn by a user to monitor changes in the user's heart rate. Pod 102 may be configured with one or more signatures 114, 105, that identify fatigue of the user. For example, one signature 105 may detect differences in the user's heart rate decay after a short burst of activity, where the change may indicate user fatigue. In another example, pod 102 includes a signature 114, 105 that detects when the user's heart rate increases without an increase in physical activity. Pod 102 may be used to monitor other conditions that cause variability of the user's heart rate, such as by detecting one or more of changes to heart rate, stride length, pace, cadence, flight time, contact time, and joint angle, and other changes to crania-caudal and other movements in the medio-lateral axis over successive strides.

### Tailored Speed Distance Monitor

In another embodiment, pod 102 is pre-loaded with a database of aggregate stride signatures 114, 105. After an initial period of usage, pod 102 connects and uploads information to PMSS 156 to indicate which signatures 114, 105 were successfully matched during usage, and to optionally upload newly learned signatures. PMSS 156 may determine, based upon knowledge of which signatures match the usage by the particular user, and download additional signatures 114, 105 to pod 102 for determining accurate speed and/or distance estimates. This approach may have several advantages: (a) more accurate measurements and estimate may be made as compared to other fitness equipment; (b) measurements and estimates are more robust, and are less susceptible to error stemming from variance in actual mounting of pod 102; (c) pod 102 may estimate power output, similar to a bicycle power meter, using calibrated and signature data; and (d) pod 102 may correct for distance variation when running on a curved or rounded track.

### Livestock Grazing Monitor

In one embodiment, pod 102 is attached to an animal to monitor the animal's location, determined using a combination of sensors & GNSS, and also to monitor other activities of the animal. For example, pod 102 may include signatures 114, 105, that determine when the animal has its head up (i.e., not feeding) and when the animal has its head down (i.e., feeding), thereby being able to determine the amount of time the animal spends feeding. Other signatures 114, 105 may be used to determine other body positions of the animal. For example, data may be collected from pods 102 attached to each animal in a herd and aggregated to estimate the amount of vegetation depleted by the herd during any given time period for a given area. The estimated vegetation consumption may be used along with other GIS data to recommend a grazing schedule in order to manage diet, environmental impact, and so on. Pods 102 may also be used to collect other data for identifying relationships among animals of the herd. For example, pods 102 may be used to identify specific animals that serve particular roles (e.g. leader vs. follower, glutton vs. abstainer, bully vs. runt).

### Ultra Long Life GNSS

In one embodiment, pod 102 includes a GNSS and positional sensors (e.g., accelerometers, gyroscopes, compass, etc.). Pod 102 makes periodic (e.g., once every two minutes) measurements using the GNSS to determine location and/or speed, and uses one or more signatures 114, 105 to estimate one or more of location, speed, and direction of movement between GNSS measurements. Pod 102 provides real-time GNSS-like positioning accuracy, while using a less power than required by devices that utilize the GNSS continually. Additionally, this approach does not suffer the effects of “position jitter” in a GNSS measurement that occur when the wireless GNSS signal is interrupted causing a constellation change.

### Blood Glucose Estimator

A diabetic monitors their blood glucose levels by frequently/regularly testing blood samples (sticking/poking). Intense activity (e.g., sport participation, rushing to catch a bus or train, etc.) may cause fast swings in blood glucose levels. Pod 102 is configured with a plurality of signatures 114, 105 that match such activity such that pod 102 may estimate when blood glucose levels are likely to change and inform or remind the diabetic when intervention may be needed. Further, pod 102 may also be configured with signatures that monitor other biometrics of the diabetic and may optionally receive information of fluid and food intake by the diabetic. Based upon matched signatures and optionally the intake, pod 102 may estimate blood glucose levels of the diabetic such that fewer blood samples may be needed. By accurately monitoring activity, biometrics, and optionally food and fluid intake, of the diabetic, using signatures 114, 105 that may be calibrated using measured blood glucose levels and known response to activity, accurate prediction of blood glucose levels may be made. Signature 114, 105 may initially be developed from a large database of similar physiques and activities, after which calibration of these signatures provide a personal response. Calibrated signatures 114, 105 may also be uploaded to PMSS 156 to form a database of signatures associated with characteristics of the diabetic.

### Self-Defined Monitor

A user of pod 102 may define their own signatures 114, 105 in a number of ways, including by recording a single motion, or repetitions of motion that are averaged, or via a graphical interface, where the user specifies which sensor(s) are used for the signature and “sketches” the signature. Such predefined signatures may not be associated with any “truth” or change of state beyond a count of the repetitions of the associated motions that have occurred. Thus, pod 102 identifies and counts repetitions of the motion defined within the signature 114, 105.

### Repetitive Stress Indication

Pod 102 may be configures with signatures that identify specific movements of the user. For example, where the user works in a machine shop that required a particularly repetitive operation, pod 102 may be configured to interrupt the user after a certain number of repetitions.

### Athlete State Indicator

Pod 102 may be configured with signatures 114 and alarms 310/409 that indicate transitions between an athlete's states. For example, pod 102 may be configured with signatures 114 and alarms 310/409 that indicate to the athlete that a warm-up period is complete. Unlike typical warm-up periods that are time based, pod 102 may determine when the athlete's activity if sufficient to have warmed the designated muscle groups of the athlete. Similarly, pod 102 may be configured to identify when the athlete has cooled down sufficiently based upon reduced, but not stopped, activity of designated muscle groups.

In another example, pod 102 is configured with signatures 114 and alarms 310/409 that alert an athlete when activity is no longer targeting a particular muscle group, or has ceased to be useful to that designated muscle group. For example, where an athlete's “form” tapers off due to fatigue, pod 102 may generate an alarm to indicate that the athlete is no longer performing satisfactorily (and may likely cause themselves an injury).

In another example, pod 102 is configured with one or more signatures and alarms 310/409 that indicate when the athlete has achieved a performance zone based upon matched signatures. For example, alarms 310/409 may be configured to provide an audible warning when the athlete's performance falls outside a specified performance zone.

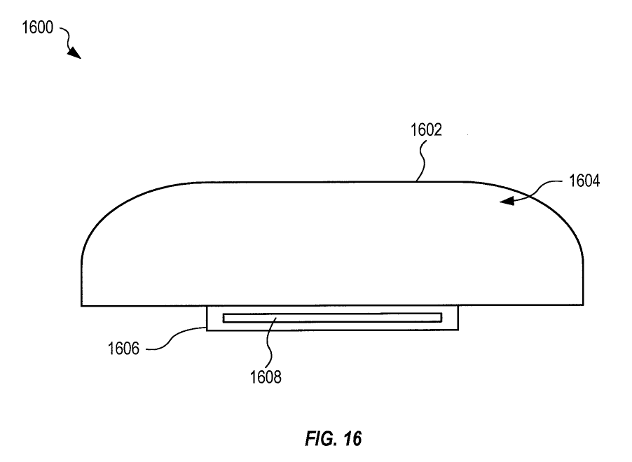

FIG. 16 shows one exemplary housing 1600 of pod 102 of FIG. 1. Housing 1600 has a shell 1602 that forms an enclosed space 1604 for containing and protecting electronics of pod 102, and an attachment loop 1606 with a slot 1608 for receiving a strap or other type of fastening to allow pod 102, within housing 1600, to be attached to a user. Housing 1600 may be attached to any one of a user's arm, wrist, leg, ankle, clothing, helmet, sporting apparatus, and part of a vehicle (e.g., bicycle, snowboard, rollerblade, and the like. Housing 1600 may be waterproof for use in wet environments.

In one embodiment, pod 102 is enclosed within a plastic enclosure that has a minimal profile that may be used as a singular unit, or may be incorporated with any one of a variety of straps, clips, and connectors. For example, pod 102 may attach to, or be incorporated within, one or more of a wrist strap, an arm band, a head band, an ankle strap, a waist strap, and a chest strap. Pod 102 may be configured to fit into a dedicated space, such as within one or more of a shoe sole, an arm band, a wrist strap, a helmet, a chest strap, and other sport and lifestyle related garments and accessories. Pod 102 may also be configured to fit into a dedicated connector that includes a clipping mechanism that may be either flexible or stiff depending on the application. The connector may be used to affix pod 102 to a user's garments, such as one or more of athletic shorts, pants, bike shorts, swimsuit, bra, headband, socks, on the inner surface of a watch or wrist strap, arm band, shoe laces, or protective equipment including shin pads, shoulder pads, helmets, and wrist guards.

The plastic enclosure of pod 102 is configured to provide a secure fit, against or close to the skin in any one of a variety of locations on the human body. Pod 102 and the selected attachment mechanism (strap/connector/clip) allows the surface of pod 102 to remain in close contact with the body of the user during use. An external surface of pod 102 may also include exterior edges and surfaces that are designed to help block and prevent ambient light from entering a PPG area for example.

The housing of pod 102 is water-resistant such that pod 102 may be worn in areas where the user is likely to sweat, or where pod 102 is exposed to external elements (e.g., rain).

### Combination of Features

Features described above as well as those claimed below may be combined in various ways without departing from the scope hereof. The following examples illustrate possible, non-limiting combinations the present invention has been described above, it should be clear that many changes and modifications may be made to the process and product without departing from the spirit and scope of this invention:

(AA) A method for determining an activity of a user, includes collecting sensor data from a plurality of sensors associated with the user, and matching, using a digital processor, the sensor data to a signature definition to determine whether the user is performing the activity. The signature definition correlated to expected sensor data from the plurality of sensor and corresponding to the activity.

(AB) In the method denoted as (AA), at least one of the plurality of sensors is located within a first pod configured to detect movement of the user.

(AC) In either of the methods denoted as (AA) or (AB), the sensors are selected from the group including: an accelerometer, a microphone, a perspiration detector, a magnetic compass, a temperature sensor, an inclination sensor, a gyroscope, an oxygen sensor, an altimeter, a short-range radar, a short range sonar, a short range laser, a pressure sensor, an image sensor, an ambient light sensor, a Global Navigation Satellite System (GNSS) receiver, an electromyogram, a signal strength detector for one or more wireless signals, an electroencephalogram, a respiration sensor, a VO2 sensor, photoplethysmograph, and an RFID receiver.

(AD) In any of the methods denoted as (AA)-(AC) the digital processor being configured with the first pod.

(AE) In any of the methods denoted as (AA)-(AD), further including receiving the signature definition from a server, wherein the signature definition is one of a plurality of signature definitions stored within a signature database and based upon expected activity of the user.

(AF) In any of the methods denoted as (AA)-(AE), further including receiving the signature definition from a second pod associated with a different user.

(AG) In any of the methods denoted as (AA)-(AE), further comprising receiving the signature definition from a second pod associated with the user.

(AH) In any of the methods denoted as (AA)-(AE), further comprising sending the signature definition to a second pod associated with a different user.

(AI) In the method denoted as (AA), the digital processor being configured with a server communicatively coupled with the first pod to receive the sensor data.

(AJ) In any of the methods denoted as (AA)-(AI), further comprising communicating from the digital processor to a user interface device that interacts with the user.

(AK) In any of the methods denoted as (AA)-(AJ), said communicating includes utilizing wireless communications.

(AL) In any of the methods denoted as (AA)-(AK), the user interface includes at least one of multi-colored LEDs, a vibrator motor, and an audio codec.

(AM) In any of the methods denoted as (AA)-(AL), further including generating an alarm based upon a matched signature definition and a predefined threshold.

(AN) In any of the methods denoted as (AA)-(AM), the signature definition comprises (a) a signature definition that defines an expected signal from at least one of the sensors when the user performs the activity, (b) a state indicative of the activity, (c) a magnitude of the activity, and (d) a truth score that indicates the accuracy of the magnitude.

(AO) In any of the methods denoted as (AA)-(AN), further including determining a state of the user based upon a history of matched signature definitions.

(AP) In any of the methods denoted as (AA)-(AO), further including determining confidence in the state based upon matched signature definition and said history.

(AQ) In any of the methods denoted as (AA)-(AP), the state includes one or more of: position, orientation, calories burned, work, level of hydration, mood, level of fatigue, heart rate, heart rate variability, skin temperature, gait type, static position, crowd flow.

(AR) In any of the methods denoted as (AA)-(AQ), further including generating a match score indicative of confidence in said matching.

(AS) In any of the methods denoted as (AA)-(AR), further including determining matched signature definition from a plurality of signature definitions based upon the match score.

(AT) In any of the methods denoted as (AA)-(AS), the sensor sensing one or more of leg motion, walking, running, skiing, skating, arm motion, gestures, heart rate, heart rate variability, wrist motion, crawling, respiration, brain waves, equipment movement, wind speed, swimming strokes, bicycle velocity, bicycle cadence, and blood glucose level.

(AU) In any of the methods denoted as (AA)-(AT), further including wirelessly sending information of the matched signature definition to a third party application running on a remote computer.

(AV) In any of the methods denoted as (AA)-(AU), the third party application utilizes an application programming interface (API) associated with the first pod that allows the third party application to communicate with the first pod to receive the information.

(AW) In any of the methods denoted as (AA)-(AV), further including generating a map within the first pod based upon matched signature definition and previously matched signature definitions, the map indicating areas navigated by the first pod.

(AX) In any of the methods denoted as (AA)-(AW), further including sending the map to a second pod to indicate, at the second pod, navigable space to a user thereof.

(AY) In any of the methods denoted as (AA)-(AX), further including receiving a map from a server associated with an area proximate the first pod to indicate at the first pod navigable space within the area.

(AZ) In any of the methods denoted as (AA)-(AY), further including using the map to validate a location of the user, determined within the first pod based upon matched signature definition.

(BA) In any of the methods denoted as (AA)-(AZ), further including calibrating the signature definition within the first pod based upon a wirelessly received direct truth measurement.

(BB) In any of the methods denoted as (AA)-(BA), further including validating matched signature definition based upon a history of recent signature definition matches.

(BC) A pod for determining activity of a user, includes a plurality of sensors capable of generating sensor data based upon sensed characteristics of the user, a memory capable of storing a signature definition based upon a known activity, a processor coupled with the memory and the sensor, a match routine, having machine readable instructions stored within the memory and executed by the processor, capable of matching the sensor data with the signature definition to determine the activity, and a transceiver capable of communicating the activity to an external device.

(BD) In the pod denoted as (BC), the sensor including one or more of an accelerometer, a gyro, a GNSS, a pressure sensor, a light sensor, and a microphone.

(BE) In either of the pods denoted as (BD) or (BE), further including an attachment device for physically coupling the pod to a part of a user's body.

(BF) In any of the pods denoted as (BC)-(BE), further including a wireless transceiver for communicating with other pods.

(BG) A system for determining when a user performs an activity includes a first pod and a server. The first pod is configured with the user and having a first sensor for generating first sensor data indicative of characteristics of the user, and a first transceiver for wirelessly transmitting the first sensor data. The server includes a processor, a memory, a second transceiver for receiving the first sensor data, a first signature definition stored within the memory and corresponding to the activity and the first sensor, and an algorithm having machine readable instructions that, when executed by the processor, are capable of matching the first sensor data to the first signature definition to determine if the user is performing the activity.

(BH) In the system denoted as (BG), further including a second pod configured with the user and including a second sensor for generating second sensor data indicative of characteristics of the user, and a third transceiver for wirelessly transmitting the second sensor data. The server further including a second signature definition stored within the memory and corresponding to the activity and the second sensor. The second transceiver is configured to receive the second sensor data and the algorithm further includes machine readable instructions that, when executed by the processor, are capable of matching the second sensor data to the second signature definition to determine if the user is performing the activity.

Changes may the above methods and systems without departing from the scope hereof. It should be made in thus be noted that the matter contained in the above description or shown in the accompanying drawings should be interpreted as illustrative and not in a limiting sense. The following claims are intended to cover all generic and specific features described herein, as well as all statements of the scope of the present method and system, which, as a matter of language, might be said to fall therebetween.

What is claimed is:

1. A method for determining an activity of a user, comprising:
collecting sensor data from a plurality of sensors associated with the user; and
matching, using a digital processor, the sensor data to a signature definition to determine whether the user is performing the activity, the signature definition correlated to expected sensor data from the plurality of sensors and corresponding to the activity.

2. The method of claim 1, wherein at least one of the plurality of sensors is located within a first pod configured to detect movement of the user.

3. The method of claim 2, wherein the sensors are selected from the group including: an accelerometer, a microphone, a perspiration detector, a magnetic compass, a temperature sensor, an inclination sensor, a gyroscope, an oxygen sensor, an altimeter, a short-range radar, a short range sonar, a short range laser, a pressure sensor, an image sensor, an ambient light sensor, a Global Navigation Satellite System (GNSS) receiver, an electromyogram, a signal strength detector for one or more wireless signals, an electroencephalogram, a respiration sensor, a VO2 sensor, photoplethysmograph, and an RFID receiver.

4. The method of claim 3, the digital processor being configured with the first pod.

5. The method of claim 4, further comprising receiving the signature definition from a server, wherein the signature definition is one of a plurality of signature definitions stored within a signature database and based upon expected activity of the user.

6. The method of claim 4, further comprising receiving the signature definition from a second pod associated with a different user.

7. The method of claim 4, further comprising receiving the signature definition from a second pod associated with the user.

8. The method of claim 4, further comprising sending the signature definition to a second pod associated with a different user.

9. The method of claim 2, the digital processor being configured with a server communicatively coupled with the first pod to receive the sensor data.

10. The method of claim 1, further comprising communicating from the digital processor to a user interface device that interacts with the user.

11. The method of claim 10, wherein said communicating comprises utilizing wireless communications.

12. The method of claim 10, wherein the user interface device comprises at least one of multi-colored LEDs, a vibrator motor, and an audio codec.

13. The method of claim 1, further comprising generating an alarm based upon a matched signature definition and a predefined threshold.

14. The method of claim 1, wherein the signature definition comprises (a) a definition of an expected signal from at least one of the sensors when the user performs the activity, (b) a state indicative of the activity, (c) a magnitude of the activity, and (d) a truth score that indicates the accuracy of the magnitude.

15. The method of claim 14, further comprising determining a state of the user based upon a history of matched signature definitions.

16. The method of claim 15, further comprising determining confidence in the state based upon matched signature definition and said history.

17. The method of claim 15, wherein the state comprises one or more of: position, orientation, calories burned, work, level of hydration, mood, level of fatigue, heart rate, heart rate variability, skin temperature, gait type, static position, crowd flow.

18. The method of claim 1, further comprising generating a match score indicative of confidence in said matching.

19. The method of claim 18, further comprising determining matched signature definition from a plurality of signature definitions based upon the match score.

20. The method of claim 1, the sensor sensing one or more of leg motion, walking, running, skiing, skating, arm motion, gestures, heart rate, heart rate variability, wrist motion, crawling, respiration, brain waves, equipment movement, wind speed, swimming strokes, bicycle velocity, bicycle cadence, and blood glucose level.

21. The method of claim 1, further comprising wirelessly sending information of the matched signature definition to a third party application running on a remote computer.

22. The method of claim 21, wherein the third party application utilizes an application programming interface (API) associated with the first pod that allows the third party application to communicate with the first pod to receive the information.

23. The method of claim 1, further comprising generating a map within the first pod based upon matched signature definition and previously matched signature definitions, the map indicating areas navigated by the first pod.

24. The method of claim 23, further comprising sending the map to a second pod to indicate, at the second pod, navigable space to a user thereof.

25. The method of claim 1, further comprising receiving a map from a server associated with an area proximate the first pod to indicate, at the first pod, navigable space within the area.

26. The method of claim 23 or 25, further comprising using the map to validate a location of the user, determined within the first pod based upon matched signature definition.

27. The method of claim 1, further comprising calibrating the signature definition within the first pod based upon a wirelessly received direct truth measurement.

28. The method of claim 1, further comprising validating matched signature definition based upon a history of recent signature definition matches.

29. A pod for determining activity of a user, comprising:
a plurality of sensors capable of generating sensor data based upon sensed characteristics of the user;
a memory capable of storing a signature definition based upon a known activity;
a processor coupled with the memory;
a match routine, comprising machine readable instructions stored within the memory and executed by the processor, capable of matching the sensor data with the signature definition to determine the activity; and
a transceiver capable of communicating the activity to an external device.

30. The pod of claim 29, the sensor comprising one or more of an accelerometer, a gyro, a GNSS, a pressure sensor, a light sensor, and a microphone.

31. The pod of claim 29, further comprising an attachment device for physically coupling the pod to a part of a user's body.

32. The pod of claim 29, further comprising a wireless transceiver for communicating with other pods.

33. A system for determining when a user performs an activity, comprising:
a first pod configured with the user and having:
a first sensor for generating first sensor data indicative of characteristics of the user; and
a first transceiver for wirelessly transmitting the first sensor data;
a server comprising:
a processor;
a memory;
a second transceiver for receiving the first sensor data;
a first signature definition stored within the memory and corresponding to the activity and the first sensor; and
an algorithm having machine readable instructions that, when executed by the processor, are capable of matching the first sensor data to the first signature definition to determine if the user is performing the activity.

34. The system of claim 33, further comprising:
a second pod configured with the user, the second pod comprising:
a second sensor for generating second sensor data indicative of characteristics of the user; and
a third transceiver for wirelessly transmitting the second sensor data; and
the server further comprising a second signature definition stored within the memory and corresponding to the activity and the second sensor;
wherein the second transceiver is configured to receive the second sensor data and wherein the algorithm further comprises machine readable instructions that, when executed by the processor, are capable of matching the second sensor data to the second signature definition to determine if the user is performing the activity.
